(19) United States
(12) Patent Application Publication
(10) Pub. No.: US 2011/0023179 A1
(43) Pub. Date: Jan. 27, 2011

(54) PLASTID TRANSIT PEPTIDES DERIVED FROM LOWER PHOTOSYNTHETIC EUKARYOTES AND METHODS

(75) Inventors: MICHAEL L. NUCCIO, DURHAM, NC (US); A. TODD RICHMOND, Raleigh, NC (US); KATERI DUNCAN, DURHAM, NC (US)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: SYNGENTA PARTICIPATIONS AG

(21) Appl. No.: 12/833,253

(22) Filed: Jul. 9, 2010

Related U.S. Application Data
(60) Provisional application No. 61/227,100, filed on Jul. 21, 2009.

(51) Int. Cl. A01H 5/00; C12N 15/82; C07K 14/405; C07H 21/04; C12N 15/63; C12N 5/10

(52) U.S. Cl. 800/278; 800/298; 800/320.1; 800/320.2; 800/312; 530/324; 536/23.1; 435/320.1; 435/419

(57) ABSTRACT

Compositions and methods for targeting polypeptides to plastids are provided. Compositions comprise plastid transit peptides as well as nucleotide sequences encoding such transit peptides and variants thereof. Compositions further comprise DNA constructs comprising a nucleotide sequence encoding the plastid transit peptide operably linked to a nucleotide sequence encoding a polypeptide of interest. These DNA constructs find use in expression and targeting of the polypeptide of interest to a plastid. Compositions also comprise expression cassettes, vectors, transformed plants, transformed plant cells, and stably transformed plant seeds wherein a polypeptide of interest is targeted to a plastid by the plastid targeting peptide of the invention.

### CROSS-REFERENCE TO RELATED APPLICATIONS

This application claims the benefit of U.S. Provisional Application No. 61/227,100, filed Jul. 21, 2009. The above application is incorporated herein by reference.

### FIELD OF THE INVENTION

The present invention relates to the genetic modification of plants, particularly to the transport of polypeptides of interest to plant plastids.

### BACKGROUND OF THE INVENTION

The plastid contains it own genome. However, during evolution many of the plastid genes were transferred to the nuclear genome. Thus, a mechanism for the transport of the nuclear encoded plastid proteins back to the plastid developed. In this manner, the protein products of these nuclear-encoded plastid genes are transferred back to the plastid after expression in the nucleus. The proteins are transported through the use of transit peptide sequences located in the N-terminus of the transported proteins. These peptides direct the proteins to the plastid and are often subsequently removed by specific proteases.

A fundamental problem in cell biology is the precise and efficient targeting of proteins synthesized by cytoplasmic ribosomes to their appropriate intracellular locations. This is especially true for transgenic higher plants where the transgene product is needed in an appropriate cellular organelle or compartment. The present invention provides a plastid targeting peptide that efficiently transports a heterologous polypeptide into the chloroplast of transgenic higher plants.

### BRIEF SUMMARY OF THE INVENTION

Compositions and methods for targeting polypeptides to chloroplasts are provided. Compositions comprise transit peptides as well as nucleotide sequences encoding a transit peptide (i.e., plastid targeting peptide or transport peptide) and variants thereof. Compositions further comprise DNA constructs comprising a nucleotide sequence encoding the transit peptide operably linked to a nucleotide sequence encoding a polypeptide of interest. These DNA constructs find use in expression and targeting of the heterologous polypeptide to a plastid. Compositions also comprise expression cassettes, vectors, transformed plants, transformed plant cells, and stably transformed plant seeds wherein a polypeptide of interest is targeted to a chloroplast by the plastid targeting peptide of the invention.

### DETAILED DESCRIPTION OF THE INVENTION

Lower photosynthetic eukaryotes include the major lineages of Chlorophytes (green algae such as Chlamydomonas and Dunaliella), Rhodophyta (red algae), Glaucocystophyta (such as Cyanophora), Euglenophyta (such as Euglena), Chlorarachniophyta, Heterokonta, Haptophyta, Cryptophyta and the dinoflagellates. The lower photosynthetic eukaryotes do not include Cyanophyta. However, the bacterial phylum Cyanophyta (cyanobacteria such as Synechococcus and Synechcystis) possess mechanisms for photosynthesis. Therefore, these photosynthetic bacteria are included as a source of basic photosynthetic mechanisms. The lower photosynthetic eukaryotes are characterized by containing a photosynthetic organelle which is evolutionarily derived from cyanobacteria. The number of membranes surrounding the photosynthetic organelle varies amongst the members of the lower photosynthetic eukaryotes. The photosynthetic organelle may be a primary plastid or a secondary (or tertiary) plastid.

Primary plastids, or simple plastids, can be found in the chlorophytes, rhodophytes and glaucocystophytes and are identified as having two bounding membranes. The organisms containing primary plastids are thought to be the only descendants of the original endosymbiotic partnership with a cyanobacterium (Harper and Keeling, Molecular Biology and Evolution 20(10): 1730-1735 (2003)). The primary plastid containing organisms have been grouped into a single supergroup referred to as the Archaeplastida (Maruyama et al BMC Evolutionary Biology 8: 151 (2008)).

Secondary (and tertiary), or complex plastids, are bounded by three or four membranes and are thought to be derived from secondary endosymbiosis wherein an organism engulfed an algae. Phylogenetic analysis of secondary and tertiary plastids suggests these plastids are derived from several different endosymbiotic events which occurred during evolution (Bhattacharya and Medlin, Plant Physiology 116: 9-15 (1998)). The euglenophytes and dinoflagellates are examples of organisms with secondary or complex plastids. See Bhattacharya and Medlin, Plant Physiology 116: 9-15 (1998) for a review. Euglenids and chlorarachniophytes have green algal secondary plastids, whereas heterokonts, haptophytes, cryptophytes and dinoflagellates all possess red algal secondary endosymbionts (Harper and Keeling, Molecular Biology and Evolution 20(10): 1730-1735 (2003)).

The plastid targeting or transport peptides of the invention mediates targeting, localization, or transport of operably linked polypeptides into plastids. Plastids are organelles found in plants and algae. Plastids are responsible for photosynthesis, storage of products such as starch, and for the synthesis of many classes of molecules such as fatty acids, terpenes, and other molecules which are needed as cellular building blocks. Plastids have the ability to differentiate, or redifferentiate, into several forms depending upon their role in the cell. Undifferentiated proplastids may develop into any of the following plastids, including, chloroplasts, chromoplasts, leucoplasts, amyloplasts, statoliths, elaioplasts, and proteinoplasts.

A “chloroplast transit peptide” or “plastid transport peptide” or “plastid transit peptide” or “transit peptide” is necessary and sufficient to facilitate the import of a protein into the plastid of its native host cell. The plastid may be a primary, secondary or tertiary plastid. The plastid may be a chloroplast. Transit peptides are located at the N-terminal end of the proteins imported into the plastids. The transit peptide facilitates co-translational or post-translational transport of an operably linked polypeptide into a plastid. These transit peptides generally comprise between 40 and 100 amino acids. Studies indicate that transit peptides contain common characteristics. These include: they are virtually devoid of negatively charged amino acids, such as aspartic acid, glutamic acid, asparagine or glutamine; the N-terminal region is devoid of charged amino acids, and of amino acids such as glycine or proline; their central region contains a very high proportion of basic or hydroxylated amino acids, such as serine or threonine; and, their C-terminal region is rich in arginine and has the ability to form an amphipathic beta-sheet secondary structure. The transit peptide is cleaved from the operably linked polypeptide, after importation, by specific proteases in the plastid.

According to one authority (Cline and Henry, Annual Review of Cellular and Developmental Biology 12: 1-26 (1996)) chloroplast transit peptides from higher plants share the following characteristics: (1) they have superficially similar properties to mitochondrial transit peptides; that is they are rich in hydroxylated residues and poor in acidic residues, (2) they are 30-120 residues long, (3) the N-terminal 10-15 amino acids are devoid of glycine, proline and charged residues, (4) the variable, middle region is rich in serine, threonine, lysine and arginine, (5) the C-proximal region contains the loosely conserved sequence (Ile/Val-x-Ala/Cys*Ala) for proteolytic processing, (6) there is no extended sequence conservation or conserved secondary structural motifs and (7) they, theoretically, adopt a predominantly random coil conformation.

Several computational approaches exist which use the above features to predict chloroplast targeting sequences in higher plants. Computational tools include PSORT (Nakai and Kanehisa, Proteins 11(2): 95-110 (1991); Horton et al., Proceedings of the 4^{th} Annual Asia Pacific Bioinformatics Conference APBC06, Taipei, Taiwan pp. 39-48 (2006); http://www.psort.org), ChloroP (Emanuelsson et al., Journal of Molecular Biology 300: 1005-1016 (1999); http://www.cbs.dtu.dk/services/ChloroP/) or TargetP (Nielsen et al., Protein Engineer 10:1-6 (1997); Emanuelsson et al., Journal of Molecular Biology 300: 1005-1016 (2000); http://www.cbs.dtu.dk/services/TargetP/).

In contrast to the higher plants, comparison of transit peptides from organisms like Chlamydomonas reinhardtii led to the following characterization: (1) they have a short, uncharged N-terminal region, (2) their central region is rich in arginine, alanine, valine and serine with a high propensity for forming an amphipathic α-helix and (3) they have a C-terminal region that may form an amphipathic β-strand (Franzen et al., FEBS Letter 260(2): 165-168 (1990)).

In one embodiment of the invention, the transit peptide is from a lower photosynthetic eukaryote which when operably linked to a heterologous protein and expressed in a transgenic higher plant, targets the heterologous protein to the chloroplast. The lower photosynthetic eukaryote is selected from the group consisting of Chlorophytes (green algae such as Chlamydomonas and Dunaliella), Rhodophyta (red algae), Glaucocystophyta (such as Cyanophora), Euglenophyta (such as Euglena), Chlorarachniophyta, Heterokonta, Haptophyta, Cryptophyta and the dinoflagellates. In another embodiment, the transit peptide is from Chlamydomonas. In another embodiment, the transit peptide is from Chlamydomonas reinhardtii. In another embodiment, the transit peptide is from Dunaliella. In another embodiment, the transit peptide is from Dunaliella salina.

Another embodiment of the invention is a method of targeting a heterologous protein to the chloroplast of a transgenic higher plant comprising the steps of operably linking a transit peptide from a lower photosynthetic eukaryote to a heterologous protein and generating a transgenic plant comprising the heterologous protein wherein the heterologous protein is detected in the chloroplast of the transgenic plant. The transgenic plant may be a higher transgenic plant. The lower photosynthetic eukaryote is selected from the group consisting of Chlorophytes (green algae such as Chlamydomonas and Dunaliella), Rhodophyta (red algae), Glaucocystophyta (such as Cyanophora), Euglenophyta (such as Euglena), Chlorarachniophyta, Heterokonta, Haptophyta, Cryptophyta and the dinoflagellates. In another embodiment, the transit peptide is from Chlamydomonas. In another embodiment, the transit peptide is from Chlamydomonas reinhardtii. In another embodiment, the transit peptide is from Dunaliella. In another embodiment, the transit peptide is from Dunaliella salina.

In one embodiment of the invention, the transit peptide is from a lower photosynthetic prokaryote which when operably linked to a heterologous protein and expressed in a transgenic higher plant, targets the heterologous protein to the chloroplast. The lower photosynthetic prokaryote is selected from the group consisting of Cyanophytes (blue-green bacteria formerly known as “blue-green algae”). In another embodiment, the transit peptide is from Synechococcus. In another embodiment, the transit peptide is from Synechococcus sp. PCC 7002. In another embodiment, the transit peptide is from Synechocystis. In another embodiment, the transit peptide is from Synechocystis sp. PCC 6803.

Another embodiment of the invention is a method of targeting a heterologous protein to the chloroplast of a transgenic higher plant comprising the steps of operably linking a transit peptide from a lower photosynthetic prokaryote to a heterologous protein and generating a transgenic plant comprising the heterologous protein wherein the heterologous protein is detected in the chloroplast of the transgenic plant. The transgenic plant may be a higher transgenic plant. The lower photosynthetic prokaryote is selected from the group consisting of Cyanophytes (blue-green bacteria formerly known as “blue-green algae”). In another embodiment, the transit peptide is from Synechococcus. In another embodiment, the transit peptide is from Synechococcus sp. PCC 7002. In another embodiment, the transit peptide is from Synechocystis. In another embodiment, the transit peptide is from Synechocystis sp. PCC 6803.

The compositions comprise nucleotide sequences encoding a transit peptide as well as variants thereof. In one embodiment the transit peptide comprises the amino acid sequence set forth in SEQ ID NOS: 2, 4, 6, 8, 10, 12, 14, 16, 18 & 20 or fragments and variants thereof as well as the nucleotide sequence set forth in SEQ ID NO: 1, 3, 5, 7, 9, 11, 13, 15, 17 & 19 or fragments and variants thereof. Compositions further comprise DNA constructs comprising a nucleotide sequence encoding the transit peptide operably linked to a nucleotide sequence encoding a heterologous polypeptide. These DNA constructs find use in expression and targeting of the heterologous polypeptide to a plastid. Compositions also comprise expression cassettes, vectors, transformed plants, transformed plant cells, and stably transformed plant seeds wherein a heterologous polypeptide is targeted to a plastid by the transit peptide of the invention.

In another embodiment of the invention, the transit peptide is from a lower photosynthetic eukaryote which when operably linked to a heterologous protein and expressed in a transgenic higher plant, targets the heterologous protein to the chloroplast. Transit peptides are derived from the nucleic acid sequence encoding a protein which is targeted to the photosynthetic organelle. Transit peptides can be found in genes known to be encoded in the nucleus of the host cell but upon translation to be targeted to the photosynthetic organelle of the host cell and can be selected from the group consisting of ferredoxin-NADP⁺-oxidoreductase, ribulose bisphosphate carboxylase/oxygenase, 5-enolpyruvyl-shikimate-3-phosphate synthase, acetolactate synthase, chloroplast ribosomal protein CS17, Cs protein, ferredoxin, plastocyanin, ribulose bisphosphate carboxylase activase, tryptophan synthase, acyl carrier protein, plastid chaperonin-60, cyochrome C552, 22-kDA heat shock protein, 33-kDA oxygen-evolving enhancer protein 1, ATP synthase gamma subunit (ATPase gamma), ATP synthase omega subunit, chlorophyll-a/b-binding proteinII-1, oxygen-evolving enhancer protein 2, oxygen-evolving enhancer protein 3, photosystem I P21, photosystem I P28, photosystem I P30, photosystem I P35, photosystem I P37, glycerol-3-phosphate acyltransferases, chlorophyll a/b binding protein, CAB2 protein, hydroxymethyl-bilane synthase, pyruvate-orthophosphate dikinase, CAB3 protein, plastid ferritin, ferritin, early light-inducible protein, glutamate-1-semialdehyde aminotransferase, protochlorophyllide reductase, starch-granule-bound amylase synthase, light-harvesting chlorophyll a/b-binding protein of photosystem II, major pollen allergen Lol p 5a, plastid ClpB ATP dependent protease, superoxide dismutase, ferredoxin NADP oxidoreductase, 28-kDa ribonucleotoprotein, 31-kDa ribonucleoprotein, 33-kDa ribonucleoprotein, acetolacate synthase, ATP synthase CFO subunit 1, 2, 3, or 4; cytochrome f, cytochrome c, ADP-glucose pyrophosphorylase, glutamine synthase, glutamine synthase 2, carbonic anhydrase, GapA protein, heat shock protein hsp 21, phosphate translocator, plastid CIpA ATP dependent protease, plastid ribosomal protein CL24, plastid ribosomal protein CL9, plastid ribosomal protein PsCL18, plastid ribosomal protein PsCL25, DAHP synthase, starch phosphorylase, root acyl carrier protein 11, betaine-aldehyde dehydrogase, GapB protein, glutamine synthase 2, phosphoribulokinase, nitrite reductase, ribosomal protein L12, ribosomal protein L13, ribosomal protein L21, ribosomal protein L35, ribosomal protein L40, triose phosphate-3-phosphoglyerate phosphate translocator, ferredoxin dependent glutamate synthase, glyceraldehyde 3 phosphate dehydrogenase, NADP dependent malic enzyme and NADP malate dehydrogenase. Table 1 identifies additional nuclear encoded genes containing plastid targeting sequences. In one embodiment of the invention the transit peptide is from Chlamydomonas reinhardtii, particularly from C. reinhardtii Photosystem I p28 and comprises the amino acid sequence set forth in SEQ ID NO: 2. In another embodiment of the invention the transit peptide is from Chlamydomonas reinhardtii, particularly from C. reinhardtii Photosystem I p30 and comprises the amino acid sequence set forth in SEQ ID NO: 4. In another embodiment of the invention the transit peptide is from Chlamydomonas reinhardtii, particularly from C. reinhardtii Photosystem I p35 and comprises the amino acid sequence set forth in SEQ ID NO: 6. In one embodiment of the invention the transit peptide is from Chlamydomonas reinhardtii, particularly from C. reinhardtii Photosystem I p37 and comprises the amino acid sequence set forth in SEQ ID NO: 8. In another embodiment of the invention the transit peptide is from Chlamydomonas reinhardtii, particularly from C. reinhardtii ssRubisco and comprises the amino acid sequence set forth in SEQ ID NO: 10. In one embodiment of the invention the transit peptide is from Chlamydomonas reinhardtii, particularly from C. reinhardtii gamma-ATPase and comprises the amino acid sequence set forth in SEQ ID NO: 12. In one embodiment of the invention the transit peptide is from cyanobacteria, particularly from Synechococcus sp. PCC 7002 cytochrome c550 and comprises the amino acid sequence set forth in SEQ ID NO: 14. In one embodiment of the invention the transit peptide is from cyanobacteria, particularly from Synechocystis sp. PCC 6803 cytochrome c553 and comprises the amino acid sequence set forth in SEQ ID NO: 16. In another embodiment of the invention the transit peptide is from cyanobacteria, particularly from Synechocystis sp. PCC 6803 psaF and comprises the amino acid sequence set forth in SEQ ID NO: 18. In another embodiment of the invention the transit peptide is from Dunaliella, particularly from Dunaliella salina EPSPS and comprises the amino acid sequence set forth in SEQ ID NO: 20.

TABLE 1
Nuclear encoded genes encoding proteins targeted to the plastid. References
are herein incorporated by reference in their entirety.

| Reference | Transit peptides | Organism |
|---|---|---|
| WO97/12983 | First paragraph, page 17 | Chlorella sorokiniana |
| | starting with the text “The | (diatom) |
| | following N-terminal | |
| | sequence . . . ” | |
| Kilian and Kroth, The Plant | FIG. 3, page 178, part “a” | Phaeodactylum |
| Journal 41: 175-183 (2005) | describing chloroplast | tricornutum (diatom) |
| | localizing signals | |
| Steiner and Loffelhardt, | FIG. 2, page 74 | Cyanophora paradoxa |
| Trends in Plant Science 7(2): | | (glaucocystophyte) |
| 72-77 (2002) | | |
| Reyes-Prieto and | Table 1, page 385 | Calvin Cycle genes |
| Bhattacharya, Molecular | | which function in the |
| Phylogentics and Evolution | | chloroplast but are |
| 45: 384-391 (2007) | | encoded in the nucleus |
| Nassoury et al, J of Cell | Materials and Methods; | Gonyaulax polyedra |
| Science 116(14): 2867-2874 | Luciferase constructs | (dinoflagellate) |
| (2003) | paragraph describing PCP | |
| | leader sequence | |
| Hackett et al, Current | Table 1, page 214 | Alexandrium tamarense |
| Biology 14: 213-218 (2004) | | (dinoflagellate) |
| Mishkind et al, J of Cell | FIG. 7, page 231 | Chlamydomonas |
| biology 100: 226-234 (1985) | | reinhardtii (green algae) |
| Martin et al, Nature 393: 162-165 | FIG. 2, genes identified in | Range of lower |
| (1998) | white boxes | photosynthetic |
| | | eukaryotes |
| Henze et al, Proc natl Acad | FIG. 2, page 9124 | Euglena gracilis (protist) |
| Sci 92: 9122-9126 (1995) | | |
| Schwartzbach et al Plant | FIG. 1, page 249 | Cyanophora paradoxa |
| Molecular Biology 38: 247-263 | | (glaucocystophyte) |
| (1998) | | |
| Steiner et al, The Plant | FIG. 1, page 647 | Cyanophora paradoxa |
| Journal 44: 646-652 (2005) | | (glaucocystophyte) |
| Kilian and Kroth, J Mol Evol | FIG. 3B, page 717 | Phaeodactylum |
| 58: 712-721 (2004) | | tricornutum (diatom) |
| Kitao et al Physiologia | FIG. 1, page 70 | Phaeodactylum |
| Plantarum 133: 68-77 (2008) | | tricornutum (diatom) |

The targeting sequences of the invention include variant or modified sequences. Such sequences have altered amino acid sequences yet retain the ability to transport a linked polypeptide into a plastid. While methods are known for substituting amino acid residues in polypeptide sequences, it is recognized that serine and threonine has been identified as abundant in transit peptides in comparison to the entire chloroplast targeting protein. Aspartic acid and glutamic acid have been reported as under represented in transit peptides. Such considerations as well as those discussed above may be taken into account when constructing variant transit peptides. However, as detailed below, one of skill in the art can assay whether a variant transit peptide is capable of effecting transport of an operably linked polypeptide for the presence of the operably linked polypeptide in the plastid. For purposes of the invention, a variant of a chloroplast transit peptide sequence of the invention also includes a fragment or fragments of the sequence.

As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

By a “crop plant” or “higher plant” is intended any plant that is cultivated for the purpose of producing plant material that is sought after by man for either oral consumption, or for utilization in an industrial, pharmaceutical, or commercial process. The invention may be applied to any of a variety of plants, including, but not limited to maize, wheat, rice, barley, soybean, cotton, sorghum, oats, tobacco, Miscanthus grass, Switch grass, trees, beans in general, rape/canola, alfalfa, flax, sunflower, safflower, millet, rye, sugarcane, sugar beet, cocoa, tea, Brassica, cotton, coffee, sweet potato, flax, peanut, clover; vegetables such as lettuce, tomato, cucurbits, cassaya, potato, carrot, radish, pea, lentils, cabbage, cauliflower, broccoli, Brussels sprouts, peppers, and pineapple; tree fruits such as citrus, apples, pears, peaches, apricots, walnuts, avocado, banana, and coconut; and flowers such as orchids, carnations, roses, and the like.

As used herein, the term “plant part” or “plant tissue” includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like.

The article “a” and “an” are used herein to refer to one or more than one (i.e., to at least one) of the grammatical object of the article. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps.

### Transit Peptide Compositions

As indicated, the compositions of the invention include transit peptides capable of transporting operably linked polypeptides into plastids. The term “peptide” broadly refers to an amino acid chain that includes naturally occurring amino acids, synthetic amino acids, genetically encoded amino acids, non-genetically encoded amino acids, and combinations thereof. Peptides can include both L-form and D-form amino acids.

Representative non-genetically encoded amino acids include but are not limited to 2-aminoadipic acid; 3-aminoadipic acid; β-aminopropionic acid; 2-aminobutyric acid; 4-aminobutyric acid (piperidinic acid); 6-aminocaproic acid; 2-aminoheptanoic acid; 2-aminoisobutyric acid; 3-aminoisobutyric acid; 2-aminopimelic acid; 2,4-diaminobutyric acid; desmosine; 2,2′-diaminopimelic acid; 2,3-diaminopropionic acid; N-ethylglycine; N-ethylasparagine; hydroxylysine; allo-hydroxylysine; 3-hydroxyproline; 4-hydroxyproline; isodesmosine; allo-isoleucine; N-methylglycine (sarcosine); N-methylisoleucine; N-methylvaline; norvaline; norleucine; and ornithine.

Representative derivatized amino acids include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups can be derivatized to form salts, methyl and ethyl esters or other types of esters or hydrazides. Free hydroxyl groups can be derivatized to form O-acyl or O-alkyl derivatives. The imidazole nitrogen of histidine can be derivatized to form N-im-benzylhistidine.

The plastid transit peptide is generally fused N-terminal to the polypeptide to be transported into a plastid. It is recognized that additional amino acid residues may be N-terminal to the plastid transit peptide. The plastid transit peptide is generally cleaved from the polypeptide of interest upon localization to the plastid. The position of cleavage may vary slightly between plant species.

The transit peptides of the invention include the transit peptide from a Chlamydomonas P28, particularly the transit peptide from a Chlamydomonas reinhardtii Photosystem I P28, more particularly the amino acid sequence set forth in SEQ ID NO: 2. The transit peptides of the invention include the transit peptide from a Chlamydomonas P30, particularly the transit peptide from a Chlamydomonas reinhardtii Photosystem I P30, more particularly the amino acid sequence set forth in SEQ ID NO: 4. The transit peptides of the invention include the transit peptide from a Chlamydomonas P35, particularly the transit peptide from a Chlamydomonas reinhardtii Photosystem I P35, more particularly the amino acid sequence set forth in SEQ ID NO: 6. The transit peptides of the invention include the transit peptide from a Chlamydomonas P37, particularly the transit peptide from a Chlamydomonas reinhardtii Photosystem I P37, more particularly the amino acid sequence set forth in SEQ ID NO: 8. The transit peptides of the invention include the transit peptide from a Chlamydomonas ssRubisco, particularly the transit peptide from a Chlamydomonas reinhardtii ssRubisco, more particularly the amino acid sequence set forth in SEQ ID NO: 10. The transit peptides of the invention include the transit peptide from a Chlamydomonas ATPase, particularly the transit peptide from a Chlamydomonas reinhardtii ATPase, more particularly the amino acid sequence set forth in SEQ ID NO: 12. The transit peptides of the invention include the transit peptide from a cyanobacteria Cytochrome c550, particularly the transit peptide from a Synechococcus sp. PCC 7002 Cytochrome c550, more particularly the amino acid sequence set forth in SEQ ID NO: 14. The transit peptides of the invention include the transit peptide from a cyanobacteria Cytochrome c553, particularly the transit peptide from a Synechocystis sp. PCC 6803 cytochrome c553, more particularly the amino acid sequence set forth in SEQ ID NO: 16. The transit peptides of the invention include the transit peptide from a Synechocystis, particularly the transit peptide from a Synechocystis sp. PCC 6803 psaF, more particularly the amino acid sequence set forth in SEQ ID NO: 18. The transit peptides of the invention include the transit peptide from a Dunaliella particularly the transit peptide from a Dunaliella salina EPSPS, more particularly the amino acid sequence set forth in SEQ ID NO: 20. Biologically active variants of the peptides of the invention are also encompassed by the present invention. Such variants should retain the ability to transport an operatively linked polypeptide into a cellular plastid. Preferably, the variant has at least the same activity as the native molecule. The ability to transport polypeptides into a plastid can be measured by methods in the art. For example, the nucleotide sequence encoding the transit peptide can be linked to a reporter gene such β-glucuronidase (GUS) or AmCyan and introduced into a plant. Analysis GUS and AmCyan activities in the subcellular fractions of the transformed plant indicate whether the transit peptide is capable of targeting the reporter proteins to the plastids. See, for example, Silva-Filho et al. (1997) Journal of Biological Chemisty 272:15264-15269 and US Patent Application No. 20080168580.

Suitable biologically active variants can be fragments and derivatives. By “fragment” is intended a peptide consisting of only a part of the intact transit peptide sequence and structure, and can be a C-terminal deletion or N-terminal deletion of amino acids or deletions at both the C- and N-terminal ends. By “derivatives” is intended any suitable modification of a transit peptide or peptide fragment encompassing any change in amino acid residues, so long as the transport activity is retained.

Peptide variants will generally have at least 70%, preferably at least 80%, more preferably about 90% to 95% or more, about 96%, about 97%, and most preferably about 98%, about 99% or more amino acid sequence identity to the amino acid sequence of the reference peptide molecule. A variant may differ by as few as 5, 4, 3, 2, or even 1 amino acid residue. Methods for determining identity between sequences are well known in the art. See, for example, the ALIGN program (Dayhoff (1978) in Atlas of Protein Sequence and Structure 5: Suppl. 3 (National Biomedical Research Foundation, Washington, D.C.) and programs in the Wisconsin Sequence Analysis Package, Version 8 (available from Genetics Computer Group, Madison, Wis.), for example, the GAP program. For purposes of optimal alignment of the two sequences, the contiguous segment of the amino acid sequence of the variant may have additional amino acid residues or deleted amino acid residues with respect to the amino acid sequence of the reference molecule. The contiguous segment used for comparison to the reference amino acid sequence will comprise at least twenty (20) contiguous nucleotides, and may be about 3, about 4, about 5, about 6, about 7, about 8, about 9, about 10, about 11, about 12, about 15, about 20, about 25, about 30, about 40 or more nucleotides. Corrections for increased sequence identity associated with inclusion of gaps in the variant's amino acid sequence can be made by assigning gap penalties. Methods of sequence alignment are well known in the art.

When considering percentage of amino acid sequence identity, some amino acid residue positions may differ as a result of conservative amino acid substitutions, which do not affect properties of protein function. In these instances, percent sequence identity may be adjusted upwards to account for the similarity in conservatively substituted amino acids. Such adjustments are well known in the art. See, for example, Myers and Miller (1988) Computer Applications in Biosciences 4:11-17.

For example, preferably, conservative amino acid substitutions may be made. A “nonessential” amino acid residue is a residue that can be altered without altering the biological activity, whereas an “essential” amino acid residue is required for biological activity. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). See, for example, Sambrook J., and Russell, D. W. (2001) Molecular Cloning: A Laboratory Manual (Cold Spring Harbor Laboratory Press, Cold Spring Harbor, N.Y.) and Innis, et al. (1990) PCR Protocols: A Guide to Methods and Applications (Academic Press, NY).

The peptides of the invention can be subject to various changes, substitutions, insertions, and deletions where such changes provide for certain advantages in its use. Thus, the term “peptide” encompasses any of a variety of forms of peptide derivatives including, for example, amides, conjugates with proteins, cyclone peptides, polymerized peptides, conservatively substituted variants, analogs, fragments, chemically modified peptides, and peptide mimetics. Any peptide that has desired transport characteristics can be used in the practice of the present invention.

By “transports,” “targets,” or “transfers” is intended that the transit peptides of the invention are capable of transporting, transferring or carrying polypeptides expressed in the nucleus of the cell into a cellular plastid. In some embodiments, the transit peptides of the invention are capable of transporting 100%, at least 90%, at least 80%, at least 70%, at least 60%, at least 50% of an operably linked polypeptide into a plastid.

One aspect of the invention pertains to isolated nucleic acid molecules comprising nucleotide sequences encoding transit peptides or biologically active portions thereof. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

Nucleic acid molecules that are fragments of these transit peptide encoding nucleotide sequences are also encompassed by the present invention. By “fragment” is intended a portion of the nucleotide sequence encoding a transit peptide. Nucleic acid molecules that are fragments of a transit peptide nucleotide sequence comprise at least about 15, about 20, about 50, about 75, about 100, about 125, about 150, about 175, about 180, about 185, about 190, about 195 contiguous nucleotides. By “contiguous” nucleotides are intended nucleotide residues that are immediately adjacent to one another.

The skilled artisan will further appreciate that changes can be introduced by mutation into the nucleotide sequences of the invention thereby leading to changes in the amino acid sequence of the encoded transit peptides, without altering the transport. Thus, variant isolated nucleic acid molecules can be created by introducing one or more nucleotide substitutions, additions, or deletions into the corresponding nucleotide sequence disclosed herein, such that one or more amino acid substitutions, additions or deletions are introduced into the encoded peptide. Mutations can be introduced by standard techniques, such as site-directed mutagenesis and PCR-mediated mutagenesis. Such variant nucleotide sequences are also encompassed by the present invention.

The compositions of the invention can be used to identify and isolate corresponding transit peptides from other organisms. In one method, the sequence can be used in hybridization assays to find sequences with substantial homology to the sequences of the invention. As used herein, the term “hybridization” is used in reference to the pairing of complementary (including partially complementary as discussed above) polynucleotide strands. Hybridization and the strength of hybridization (i.e., the strength of the association between polynucleotide strands) is impacted by many factors well known in the art including the degree of complementarity between the polynucleotides; stringency of the conditions involved that are affected by such conditions as the concentration of salts; the melting temperature (Tm) of the formed hybrid; the presence of other components; the molarity of the hybridizing strands; and, the G:C content of the polynucleotide strands.

Thus, isolated sequences that are capable of transporting polypeptides into a plastid and which hybridize under stringent conditions to the sequences disclosed herein, or to fragments thereof, are encompassed by the present invention. Such sequences will be at least about 40% to 50% homologous, about 60%, 65%, or 70% homologous, and even at least about 75%, 80%, 85%, 90%, 91%, 92%, 93%, 94%, 95%, 96%, 97%, 98%, 99% or more homologous with the disclosed sequences. That is, the sequence identity of sequences may range, sharing at least about 40% to 50%, about 60%, 65%, or 70%, and even at least about 75%, 80%, 85%, 90%, 91%, 92%, 93%, 94%, 95%, 96%, 97%, 98%, 99% or more sequence identity.

As used herein, “Tm” and “melting temperature” are interchangeable terms which are the temperature at which 50% of a population of double-stranded polynucleotide molecules becomes dissociated into single strands. The equation for calculating the Tm of polynucleotides is well known in the art. For example, the Tm may be calculated by the following equation: Tm=69.3+0.41×(G+C) %−650/L, wherein L is the length of the probe in nucleotides. The Tm of a hybrid polynucleotide may also be estimated using a formula adopted from hybridization assays in 1 M salt, and commonly used for calculating Tm for PCR primers: [(number of A+T)×2° C.+(number of G+C)×4° C.], see, for example, Newton et al. (1997) PCR 2nd Ed. (Springer-Verlag, New York). Other more sophisticated computations exist in the art, which take structural as well as sequence characteristics into account for the calculation of Tm. A calculated Tm is merely an estimate; the optimum temperature is commonly determined empirically.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

Corresponding sequences can also be identified by PCR reactions. See, Sambrook et al. (1989) Molecular Cloning: A Laboratory Manual (2d ed., Cold Spring Harbor Laboratory Press, Cold Spring Harbor, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). The primers of the present invention can be prepared using techniques known in the art, including, but not limited to, cloning and digestion of the appropriate sequences and direct chemical synthesis.

Chemical synthesis methods that can be used to make the primers of the present invention, include, but are not limited to, the phosphotriester method described by Narang et al., Methods in Enzymology, 68:90 (1979), the phosphodiester method disclosed by Brown et al., Methods in Enzymology, 68:109 (1979), the diethylphosphoramidate method disclosed by Beaucage et al., Tetrahedron Letters, 22:1859 (1981) and the solid support method described in U.S. Pat. No. 4,458,066. The use of an automated oligonucleotide synthesizer to prepare synthetic oligonucleotide primers of the present invention is also contemplated herein. Additionally, if desired, the primers can be labeled using techniques known in the art and described below.

### Plant Expression Cassettes

As discussed, the plastid targeting peptides of the invention operably linked to a polypeptide of interest transports the linked polypeptide into a plastid. Thus, when assembled into a DNA construct and used to transform a plant, the targeting peptide directs a polypeptide of interest to plastids in the transformed plant cells, plants, and seeds after expression. Thus, the compositions of the invention also comprise nucleic acid sequences for transformation and expression and accumulation of a polypeptide of interest in the plastids of a plant cell of interest. The nucleic acid sequences may be present in DNA constructs or expression cassettes. “Expression cassette” as used herein means a nucleic acid molecule capable of directing expression of a particular nucleotide sequence in an appropriate host cell, comprising a promoter operatively linked to the nucleotide sequence of interest (i.e., a nucleotide sequence encoding a polypeptide of interest) which is operatively linked to termination signals. It also typically comprises sequences required for proper translation of the nucleotide sequence. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. Typically, however, the expression cassette is heterologous with respect to the host, i.e., the particular DNA sequence of the expression cassette does not occur naturally in the host cell and must have been introduced into the host cell or an ancestor of the host cell by a transformation event. The expression of the nucleotide sequence in the expression cassette may be under the control of a constitutive promoter or of an inducible promoter that initiates transcription only when the host cell is exposed to some particular external stimulus. Additionally, the promoter can also be specific to a particular tissue or organ or stage of development.

The present invention encompasses the transformation of plants with expression cassettes capable of directing expression and accumulation of a polypeptide of interest in the plastids of a plant cell. The expression cassette will include in the 5′-3′ direction of transcription, a transcriptional and translational initiation region (i.e., a promoter), a polynucleotide encoding a plastid targeting peptide, and a polynucleotide encoding a polypeptide of interest. The expression cassette may optionally comprise a transcriptional and translational termination region (i.e. termination region) functional in plants.

In addition to the polynucleotide sequence encoding the plastid targeting peptide, the construct may further comprise additional regulatory elements to facilitate transcription, translation, or transport of the polypeptide of interest. The regulatory sequences of the expression construct are operably linked to the polynucleotide of interest. By “operably linked” is intended a functional linkage between a regulatory element and a second sequence wherein the regulatory element initiates and/or mediates transcription, translation, or translocation of the DNA sequence corresponding to the second sequence. Generally, the term “operably linked” means that the nucleotide sequences being linked are contiguous or adjacent to one another. The regulatory elements include promoters, enhancers, and signal sequences useful for targeting cytoplasmically-synthesized proteins to plastids of the plant cell. In one embodiment, the construct comprises, in the 5′ to 3′ direction of transcription, a transcriptional and translational initiation region (i.e., a promoter), a polynucleotide encoding a plastid targeting peptide, and a polynucleotide encoding a polypeptide of interest.

Any promoter capable of driving expression in the plant of interest may be used in the practice of the invention. The promoter may be native or analogous or foreign or heterologous to the plant host. The terms “heterologous” and “exogenous” when used herein to refer to a nucleic acid sequence (e.g. a DNA or RNA sequence) or a gene, refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides.

A “homologous” nucleic acid (e.g. DNA) sequence is a nucleic acid (e.g. DNA or RNA) sequence naturally associated with a host cell into which it is introduced.

The choice of promoters to be included depends upon several factors, including, but not limited to, efficiency, selectability, inducibility, desired expression level, and cell- or tissue-preferential expression. It is a routine matter for one of skill in the art to modulate the expression of a sequence by appropriately selecting and positioning promoters and other regulatory regions relative to that sequence.

Some suitable promoters initiate transcription only, or predominantly, in certain cell types. Thus, as used herein a cell type- or tissue-preferential promoter is one that drives expression preferentially in the target tissue, but may also lead to some expression in other cell types or tissues as well. Methods for identifying and characterizing promoter regions in plant genomic DNA include, for example, those described in the following references: Jordano, et al., Plant Cell, 1:855-866 (1989); Bustos, et al., Plant Cell, 1:839-854 (1989); Green, et al., EMBO Journal 7, 4035-4044 (1988); Meier, et al., Plant Cell, 3, 309-316 (1991); and Zhang, et al., Plant Physiology 110: 1069-1079 (1996).

Promoters active in photosynthetic tissue in order to drive transcription in green tissues such as leaves and stems are of particular interest for the present invention. Most suitable are promoters that drive expression only or predominantly in such tissues. The promoter may confer expression constitutively throughout the plant, or differentially with respect to the green tissues, or differentially with respect to the developmental stage of the green tissue in which expression occurs, or in response to external stimuli.

Examples of such promoters include the ribulose-1,5-bisphosphate carboxylase (RbcS) promoters such as the RbcS promoter from eastern larch (Larix laricina), the pine cab6 promoter (Yamamoto et al. (1994) Plant and Cell Physiology 35:773-778), the Cab-1 gene promoter from wheat (Fejes et al. (1990) Plant Molecular Biology 15:921-932), the CAB-1 promoter from spinach (Lubberstedt et al. (1994) Plant Physiology 104:997-1006), the cab1R promoter from rice (Luan et al. (1992) Plant Cell 4:971-981), the pyruvate orthophosphate dikinase (PPDK) promoter from corn (Matsuoka et al. (1993) PNAS USA 90:9586-9590), the tobacco Lhcb1*2 promoter (Cerdan et al. (1997) Plant Molecular Biology 33:245-255), the Arabidopsis thaliana SUC2 sucrose-H+ symporter promoter (Truernit et al. (1995) Planta 196:564-570), and thylakoid membrane protein promoters from spinach (psaD, psaF, psaE, PC, FNR, atpC, atpD, cab, rbcS. Other promoters that drive transcription in stems, leafs and green tissue are described in U.S. Patent Publication No. 2007/0006346, herein incorporated by reference in its entirety.

A maize gene encoding phosphoenol carboxylase (PEPC) has been described by Hudspeth & Grula (Plant Molecular Biology 12: 579-589 (1989)). Using standard molecular biological techniques the promoter for this gene can be used to drive the expression of any gene in a green tissue-specific manner in transgenic plants.

In some other embodiments of the present invention, inducible promoters may be desired. Inducible promoters drive transcription in response to external stimuli such as chemical agents or environmental stimuli. For example, inducible promoters can confer transcription in response to hormones such as giberellic acid or ethylene, or in response to light or drought.

A variety of transcriptional terminators are available for use in expression cassettes. These are responsible for the termination of transcription beyond the transgene and correct mRNA polyadenylation. The termination region may be native with the transcriptional initiation region, may be native with the operably linked DNA sequence of interest, may be native with the plant host, or may be derived from another source (i.e., foreign or heterologous to the promoter, the DNA sequence of interest, the plant host, or any combination thereof). Appropriate transcriptional terminators are those that are known to function in plants and include the CAMV 35S terminator, the tml terminator, the nopaline synthase terminator and the pea rbcs E9 terminator. These can be used in both monocotyledons and dicotyledons. In addition, a gene's native transcription terminator may be used.

In some embodiments, the expression cassette will comprise a selectable marker gene for the selection of transformed cells. Selectable marker genes are utilized for the selection of transformed cells or tissues.

Numerous sequences have been found to enhance gene expression from within the transcriptional unit and these sequences can be used in conjunction with the genes of this invention to increase their expression in transgenic plants.

Various intron sequences have been shown to enhance expression, particularly in monocotyledonous cells. For example, the introns of the maize Adhl gene have been found to significantly enhance the expression of the wild-type gene under its cognate promoter when introduced into maize cells. Intron 1 was found to be particularly effective and enhanced expression in fusion constructs with the chloramphenicol acetyltransferase gene (Callis et al., Genes & Development 1: 1183-1200 (1987)). In the same experimental system, the intron from the maize bronze 1 gene had a similar effect in enhancing expression. Intron sequences have been routinely incorporated into plant transformation vectors, typically within the non-translated leader.

A number of non-translated leader sequences derived from viruses are also known to enhance expression, and these are particularly effective in dicotyledonous cells. Specifically, leader sequences from Tobacco Mosaic Virus (TMV, the “W-sequence”), Maize Chlorotic Mottle Virus (MCMV), and Alfalfa Mosaic Virus (AMV) have been shown to be effective in enhancing expression (e.g. Gallie et al. Nucleic Acids Research 15: 8693-8711 (1987); Skuzeski et al. Plant Molecular Biology 15: 65-79 (1990)). Other leader sequences known in the art include but are not limited to: picornavirus leaders, for example, EMCV leader (Encephalomyocarditis 5′ noncoding region) (Elroy-Stein, O., Fuerst, T. R., and Moss, B. PNAS USA 86:6126-6130 (1989)); potyvirus leaders, for example, TEV leader (Tobacco Etch Virus) (Allison et al., 1986); MDMV leader (Maize Dwarf Mosaic Virus); Virology 154:9-20); human immunoglobulin heavy-chain binding protein (BiP) leader, (Macejak, D. G., and Samow, P., Nature 353: 90-94 (1991); untranslated leader from the coat protein mRNA of alfalfa mosaic virus (AMV RNA 4), (Jobling, S. A., and Gehrke, L., Nature 325:622-625 (1987); tobacco mosaic virus leader (TMV), (Gallie, D. R. et al., Molecular Biology of RNA, pages 237-256 (1989); and Maize Chlorotic Mottle Virus leader (MCMV) (Lommel, S. A. et al., Virology 81:382-385 (1991). See also, Della-Cioppa et al., Plant Physiology 84:965-968 (1987).

As indicated the plastid transit peptide is operably linked to a polypeptide to be transported into the plastid. Polypeptides for traits of interest include agronomic traits that primarily are of benefit to a seed company, a grower, or a grain processor, for example, herbicide resistance, virus resistance, bacterial pathogen resistance, insect resistance, nematode resistance, and fungal resistance. See, e.g., U.S. Pat. Nos. 5,569,823; 5,304,730; 5,495,071; 6,329,504; and 6,337,431. Traits of interest may also be one that increases plant vigor or yield (including traits that allow a plant to grow at different temperatures, soil conditions and levels of sunlight and precipitation), or one that allows identification of a plant exhibiting a trait of interest (e.g., selectable marker gene, seed coat color, etc.). A plethora of genes useful for generating plants with desired secondary traits are available in the art.

Herbicide resistance genes of interest include those encoding precursors of acetolactase synthetase (ALS), see, for example, U.S. Pat. No. 5,013,659; mutated acetolactate synthetase; 3-enolpyruvylshikimate-5-phosphate synthetase (EPSP synthetase), see, for example, U.S. Pat. Nos. 4,971,908 and 6,225,114; enzymes that modify a physiological process that occurs in a plastid including photosynthesis, fatty acid, amino acid, oil, arotenoid, terpenoid, and starch; etc. Other genes of interest include but are not limited to those encoding zeaxanthin epoxidase, choline monooxygenase, ferrochelatase, omega-3 fatty acid desaturase, glutamine synthetase, starch modifying enzymes, essential amino acids, provitamin A, hormones, Bt toxin proteins, and the like. Nucleotide sequences for such polypeptides are available in the art.

### Plants

Plants useful in the present invention include plants that are transgenic for at least a polynucleotide encoding a plastid-targeted polypeptide of interest. Such plants will comprise a polynucleotide encoding a targeting peptide operably linked to a polynucleotide encoding a polypeptide of interest. One of skill in the art will recognize that the polypeptide sequences of interest may be associated with or contributing to one or more secondary trait(s) of interest. These polypeptides are cytoplasmically-expressed, and targeted to a plastid.

The type of plant selected depends on a variety of factors, including for example, the downstream use of the harvested plant material, amenability of the plant species to transformation, and the conditions under which the plants will be grown, harvested, and/or processed. One of skill in the art will further recognize that additional factors for selecting appropriate plant varieties for use in the present invention include high yield potential, good stalk strength, resistance to specific diseases, drought tolerance, rapid dry down and grain quality sufficient to allow storage and shipment to market with minimum loss.

It is further contemplated that the constructs of the invention may be introduced into plant varieties having improved properties suitable or optimal for a particular downstream use. Plants useful in the present invention include, but are not limited to, monocotyledonous and dicotyledonous plants, particularly crop plants such as maize, soybean, wheat, rice, Brassica, and the like. Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), cotton (Gossypium barbadense, Gossypium hirsutum), sweet potato (Ipomoea batatus), cassaya (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Plants of interest include cauliflower (Brassica oleracea), artichoke (Cynara scolvmus), and safflower (Carthamus, e.g. tinctorius); fruits such as apple (Malus, e.g. domesticus), banana (Musa, e.g. acuminata), berries (such as the currant, Ribes, e.g. rubrum), cherries (such as the sweet cherry, Prunus, e.g. avium), cucumber (Cucumis, e.g. sativus), grape (Vitis, e.g. vinifera), lemon (Citrus limon), melon (Cucumis melo), nuts (such as the walnut, Juglans, e.g. regia; peanut, Arachis hypoaeae), orange (Citrus, e.g. maxima), peach (Prunus, e.g. persica), pear (Pyra, e.g. communis), pepper (Solanum, e.g. capsicum), plum (Prunus, e.g. domestica), strawberry (Fragaria, e.g. moschata), tomato (Lycopersicon, e.g. esculentum); leafs, such as alfalfa (Medicago, e.g. sativa), sugar cane (Saccharum), cabbages (such as Brassica oleracea), endive (Cichoreum, e.g. endivia), leek (Allium, e.g. porrum), lettuce (Lactuca, e.g. sativa), spinach (Spinacia e.g. oleraceae), tobacco (Nicotiana, e.g. tabacum); roots, such as arrowroot (Maranta, e.g. arundinacea), beet (Beta, e.g. vulgaris), carrot (Daucus, e.g. carota), cassaya (Manihot, e.g. esculenta), turnip (Brassica, e.g. rapa), radish (Raphanus, e.g. sativus) yam (Dioscorea, e.g. esculenta), sweet potato (Ipomoea batatas); seeds, such as bean (Phaseolus, e.g. vulgaris), pea (Pisum, e.g. sativum), soybean (Glycine, e.g. max), wheat (Triticum, e.g. aestivum), barley (Hordeum, e.g. vulgare), corn (Zea, e.g. mays), rice (Oryza, e.g. sativa); grasses, such as Miscanthus grass (Miscanthus, e.g., giganteus) and switchgrass (Panicum, e.g. virgatum); trees such as poplar (Populus, e.g. tremula), pine (Pinus); shrubs, such as cotton (e.g., Gossypium hirsutum); and tubers, such as kohlrabi (Brassica, e.g. oleraceae), potato (Solanum, e.g. tuberosum), and the like.

### Plant Transformation

The expression constructs described herein can be introduced into the plant cell in a number of art-recognized ways. The term “introducing” in the context of a polynucleotide, for example, a nucleotide construct of interest, is intended to mean presenting to the plant the polynucleotide in such a manner that the polynucleotide gains access to the interior of a cell of the plant. Where more than one polynucleotide is to be introduced, these polynucleotides can be assembled as part of a single nucleotide construct, or as separate nucleotide constructs, and can be located on the same or different transformation vectors. Accordingly, these polynucleotides can be introduced into the host cell of interest in a single transformation event, in separate transformation events, or, for example, in plants, as part of a breeding protocol. The methods of the invention do not depend on a particular method for introducing one or more polynucleotides into a plant, only that the polynucleotide(s) gains access to the interior of at least one cell of the plant. Methods for introducing polynucleotides into plants are known in the art including, but not limited to, transient transformation methods, stable transformation methods, and virus-mediated methods.

“Transient transformation” in the context of a polynucleotide is intended to mean that a polynucleotide is introduced into the plant and does not integrate into the genome of the plant.

By “stably introducing” or “stably introduced” in the context of a polynucleotide introduced into a plant is intended the introduced polynucleotide is stably incorporated into the plant genome, and thus the plant is stably transformed with the polynucleotide.

“Stable transformation” or “stably transformed” is intended to mean that a polynucleotide, for example, a nucleotide construct described herein, introduced into a plant integrates into the genome of the plant and is capable of being inherited by the progeny thereof, more particularly, by the progeny of multiple successive generations.

Numerous transformation vectors available for plant transformation are known to those of ordinary skill in the plant transformation arts, and the genes pertinent to this invention can be used in conjunction with any such vectors. The selection of vector will depend upon the preferred transformation technique and the target species for transformation. For certain target species, different antibiotic or herbicide selection markers may be preferred. Selection markers used routinely in transformation include the nptII gene, which confers resistance to kanamycin and related antibiotics (Messing & Vierra. Gene 19: 259-268 (1982); Bevan et al., Nature 304:184-187 (1983)), the bar gene, which confers resistance to the herbicide phosphinothricin (White et al., Nucleic Acids Research 18: 1062 (1990), Spencer et al. Theoretical and Applied Genetics 79: 625-631 (1990)), the hph gene, which confers resistance to the antibiotic hygromycin (Blochinger & Diggelmann, Molecular and Cellular Biology 4: 2929-2931), and the dhfr gene, which confers resistance to methatrexate (Bourouis et al., EMBO Journal 2(7): 1099-1104 (1983)), the EPSPS gene, which confers resistance to glyphosate (U.S. Pat. Nos. 4,940,935 and 5,188,642), and the mannose-6-phosphate isomerase gene, which provides the ability to metabolize mannose (U.S. Pat. Nos. 5,767,378 and 5,994,629).

Methods for regeneration of plants are also well known in the art. For example, Ti plasmid vectors have been utilized for the delivery of foreign DNA, as well as direct DNA uptake, liposomes, electroporation, microinjection, and microprojectiles. In addition, bacteria from the genus Agrobacterium can be utilized to transform plant cells. Below are descriptions of representative techniques for transforming both dicotyledonous and monocotyledonous plants, as well as a representative plastid transformation technique.

Many vectors are available for transformation using Agrobacterium tumefaciens. These typically carry at least one T-DNA border sequence and include vectors such as pBIN19 (Bevan, Nucleic Acids Research (1984)). For the construction of vectors useful in Agrobacterium transformation, see, for example, US Patent Application Publication No. 2006/0260011, herein incorporated by reference.

Transformation without the use of Agrobacterium tumefaciens circumvents the requirement for T-DNA sequences in the chosen transformation vector and consequently vectors lacking these sequences can be utilized in addition to vectors such as the ones described above which contain T-DNA sequences. Transformation techniques that do not rely on Agrobacterium include transformation via particle bombardment, protoplast uptake (e.g. PEG and electroporation) and microinjection. The choice of vector depends largely on the preferred selection for the species being transformed.

Transformation techniques for dicotyledons are well known in the art and include Agrobacterium-based techniques and techniques that do not require Agrobacterium. Non-Agrobacterium techniques involve the uptake of exogenous genetic material directly by protoplasts or cells. This can be accomplished by PEG or electroporation mediated uptake, particle bombardment-mediated delivery, or microinjection. Examples of these techniques are described by Paszkowski et al., EMBO Journal 3: 2717-2722 (1984), Potrykus et al., Molecular Genetics and Genomics 199: 169-177 (1985), Reich et al., Biotechnology 4: 1001-1004 (1986), and Klein et al., Nature 327: 70-73 (1987). In each case the transformed cells are regenerated to whole plants using standard techniques known in the art.

Agrobacterium-mediated transformation is a preferred technique for transformation of dicotyledons because of its high efficiency of transformation and its broad utility with many different species. Agrobacterium transformation typically involves the transfer of the binary vector carrying the foreign DNA of interest (e.g. pCIB200 or pCIB2001) to an appropriate Agrobacterium strain which may depend of the complement of vir genes carried by the host Agrobacterium strain either on a co-resident Ti plasmid or chromosomally (e.g. strain CIB542 for pCIB200 and pCIB2001 (Uknes et al. Plant Cell 5: 159-169 (1993)). The transfer of the recombinant binary vector to Agrobacterium is accomplished by a triparental mating procedure using E. coli carrying the recombinant binary vector, a helper E. coli strain which carries a plasmid such as pRK2013 and which is able to mobilize the recombinant binary vector to the target Agrobacterium strain. Alternatively, the recombinant binary vector can be transferred to Agrobacterium by DNA transformation (Hofgen & Willmitzer, Nucleic Acids Research 16: 9877 (1988)).

Transformation of the target plant species by recombinant Agrobacterium usually involves co-cultivation of the Agrobacterium with explants from the plant and follows protocols well known in the art. Transformed tissue is regenerated on selectable medium carrying the antibiotic or herbicide resistance marker present between the binary plasmid T-DNA borders.

Another approach to transforming plant cells with a gene involves propelling inert or biologically active particles at plant tissues and cells. This technique is disclosed in U.S. Pat. Nos. 4,945,050, 5,036,006, and 5,100,792 all to Sanford et al. Generally, this procedure involves propelling inert or biologically active particles at the cells under conditions effective to penetrate the outer surface of the cell and afford incorporation within the interior thereof. When inert particles are utilized, the vector can be introduced into the cell by coating the particles with the vector containing the desired gene. Alternatively, the target cell can be surrounded by the vector so that the vector is carried into the cell by the wake of the particle. Biologically active particles (e.g., dried yeast cells, dried bacterium or a bacteriophage, each containing DNA sought to be introduced) can also be propelled into plant cell tissue.

Transformation of most monocotyledon species has now also become routine. Preferred techniques include direct gene transfer into protoplasts using PEG or electroporation techniques, and particle bombardment into callus tissue. Transformations can be undertaken with a single DNA species or multiple DNA species (i.e. co-transformation) and both of these techniques are suitable for use with this invention. Co. transformation may have the advantage of avoiding complete vector construction and of generating transgenic plants with unlinked loci for the gene of interest and the selectable marker, enabling the removal of the selectable marker in subsequent generations, should this be regarded desirable. However, a disadvantage of the use of co-transformation is the less than 100% frequency with which separate DNA species are integrated into the genome (Schocher et al. Biotechnology 4: 1093-1096 (1986)).

Patent Applications EP 0 292 435, EP 0 392 225, and WO 93/07278 describe techniques for the preparation of callus and protoplasts from an elite inbred line of maize, transformation of protoplasts using PEG or electroporation, and the regeneration of maize plants from transformed protoplasts. Gordon-Kamm et al. (Plant Cell 2: 603-618 (1990)) and Fromm et al. (Biotechnology 8: 833-839 (1990)) have published techniques for transformation of A188-derived maize line using particle bombardment. Furthermore, WO 93/07278 and Koziel et al. (Biotechnology 11: 194-200 (1993)) describe techniques for the transformation of elite inbred lines of maize by particle bombardment. This technique utilizes immature maize embryos of 1.5-2.5 mm length excised from a maize ear 14-15 days after pollination and a PDS-1000He Biolistics device for bombardment.

Transformation of rice can also be undertaken by direct gene transfer techniques utilizing protoplasts or particle bombardment. Protoplast-mediated transformation has been described for Japonica-types and Indica-types (Zhang et al. Plant Cell Reports 7: 379-384 (1988); Shimamoto et al. Nature 338: 274-277 (1989); Datta et al. Biotechnology 8: 736-740 (1990)). Both types are also routinely transformable using particle bombardment (Christou et al. Biotechnology 9: 957-962 (1991)). Furthermore, WO 93/21335 describes techniques for the transformation of rice via electroporation.

Patent Application EP 0 332 581 describes techniques for the generation, transformation and regeneration of Pooideae protoplasts. These techniques allow the transformation of Dactylis and wheat. Furthermore, wheat transformation has been described by Vasil et al. (Biotechnology 10: 667-674 (1992)) using particle bombardment into cells of type C long-term regenerable callus, and also by Vasil et al. (Biotechnology 11:1553-1558 (1993)) and Weeks et al. (Plant Physiology 102: 1077-1084 (1993)) using particle bombardment of immature embryos and immature embryo-derived callus. A preferred technique for wheat transformation, however, involves the transformation of wheat by particle bombardment of immature embryos and includes either a high sucrose or a high maltose step prior to gene delivery. Prior to bombardment, any number of embryos (0.75-1 mm in length) are plated onto MS medium with 3% sucrose (Murashiga & Skoog, Physiologia Plantarum 15: 473-497 (1962)) and 3 mg/L 2,4-D for induction of somatic embryos, which is allowed to proceed in the dark. On the chosen day of bombardment, embryos are removed from the induction medium and placed onto the osmoticum (i.e. induction medium with sucrose or maltose added at the desired concentration, typically 15%). The embryos are allowed to plasmolyze for 2-3 hours and are then bombarded. Twenty embryos per target plate are typical, although not critical. An appropriate gene-carrying plasmid is precipitated onto micrometer size gold particles using standard procedures. Each plate of embryos is shot with the DuPont BIOLISTICS® helium device using a burst pressure of about 1000 psi using a standard 80 mesh screen. After bombardment, the embryos are placed back into the dark to recover for about 24 hours, still on osmotic media. After 24 hours, the embryos are removed from the osmoticum and placed back onto induction medium where they stay for about a month before regeneration. Approximately one month later the embryo explants with developing embryogenic callus are transferred to regeneration medium (MS+1 mg/L NAA, 5 mg/LGA), further containing the appropriate selection agent. After approximately one month, developed shoots are transferred to larger sterile containers known as “GA7s” which contain half-strength MS, 2% sucrose, and the same concentration of selection agent.

Transformation of monocotyledons using Agrobacterium has also been described. See, WO 94/00977 and U.S. Pat. No. 5,591,616, and, Negrotto et al., Plant Cell Reports 19: 798-803 (2000).

For example, rice (Oryza sativa) can be used for generating transgenic plants. Various rice cultivars can be used (Hiei et al., 1994, Plant Journal 6:271-282; Dong et al., 1996, Molecular Breeding 2:267-276; Hiei et al., 1997, Plant Molecular Biology, 35:205-218). Also, the various media constituents described below may be either varied in quantity or substituted. Embryogenic responses are initiated and/or cultures are established from mature embryos by culturing on MS-CIM medium (MS basal salts, 4.3 g/L; B5 vitamins (200×), 5 ml/L; Sucrose, 30 g/L; proline, 500 mg/L; glutamine, 500 mg/L; casein hydrolysate, 300 mg/L; 2,4-D (1 mg/ml), 2 ml/L; adjust pH to 5.8 with 1 N KOH; Phytagel, 3 g/L). Either mature embryos at the initial stages of culture response or established culture lines are inoculated and co-cultivated with the Agrobacterium tumefaciens strain LBA4404 (Agrobacterium) containing the desired vector construction. Agrobacterium is cultured from glycerol stocks on solid YPC medium (100 mg/L spectinomycin and any other appropriate antibiotic) for about 2 days at 28° C. Agrobacterium is re-suspended in liquid MS-CIM medium. The Agrobacterium culture is diluted to an OD600 of 0.2-0.3 and acetosyringone is added to a final concentration of 200 uM. Acetosyringone is added before mixing the solution with the rice cultures to induce Agrobacterium for DNA transfer to the plant cells. For inoculation, the plant cultures are immersed in the bacterial suspension. The liquid bacterial suspension is removed and the inoculated cultures are placed on co-cultivation medium and incubated at 22° C. for two days. The cultures are then transferred to MS-CIM medium with Ticarcillin (400 mg/L) to inhibit the growth of Agrobacterium. For constructs utilizing the PMI selectable marker gene (Reed et al., In Vitro Cellular & Developmental Biology-Plant 37:127-132), cultures are transferred to selection medium containing Mannose as a carbohydrate source (MS with 2% Mannose, 300 mg/L Ticarcillin) after 7 days, and cultured for 3-4 weeks in the dark. Resistant colonies are then transferred to regeneration induction medium (MS with no 2,4-D, 0.5 mg/L IAA, 1 mg/L zeatin, 200 mg/Ltimentin 2% Mannose and 3% Sorbitol) and grown in the dark for 14 days. Proliferating colonies are then transferred to another round of regeneration induction media and moved to the light growth room. Regenerated shoots are transferred to GA7 containers with GA7-1 medium (MS with no hormones and 2% Sorbitol) for 2 weeks and then moved to the greenhouse when they are large enough and have adequate roots. Plants are transplanted to soil in the greenhouse (T0 generation) grown to maturity, and the T1 seed is harvested.

The plants obtained via transformation with a nucleic acid sequence of the present invention can be any of a wide variety of plant species, including those of monocots and dicots; however, the plants used in the method of the invention are preferably selected from the list of agronomically important target crops set forth supra. The expression of a gene of the present invention in combination with other characteristics important for production and quality can be incorporated into plant lines through breeding. Breeding approaches and techniques are known in the art. See, for example, Welsh J. R., Fundamentals of Plant Genetics and Breeding, John Wiley & Sons, NY (1981); Crop Breeding, Wood D. R. (Ed.) American Society of Agronomy Madison, Wis. (1983); Mayo O., The Theory of Plant Breeding, Second Edition, Clarendon Press, Oxford (1987); Singh, D. P., Breeding for Resistance to Diseases and Insect Pests, Springer-Verlag, NY (1986); and Wricke and Weber, Quantitative Genetics and Selection Plant Breeding, Walter de Gruyter and Co., Berlin (1986).

For the transformation of plastids, seeds of Nicotiana tabacum c.v. “Xanthienc” are germinated seven per plate in a 1″ circular array on T agar medium and bombarded 12-14 days after sowing with 1 um tungsten particles (M10, Biorad, Hercules, Calif.) coated with DNA from plasmids pPH143 and pPH145 essentially as described (Svab, Z. and Maliga, P. (1993) PNAS USA 90, 913-917). Bombarded seedlings are incubated on T medium for two days after which leaves are excised and placed abaxial side up in bright light (350-500 umol photons/m²/s) on plates of RMOP medium (Svab, Z., Hajdukiewicz, P. and Maliga, P. (1990) PNAS USA 87, 8526-8530) containing 500 μg/ml spectinomycin dihydrochloride (Sigma, St. Louis, Mo.). Resistant shoots appearing underneath the bleached leaves three to eight weeks after bombardment are subcloned onto the same selective medium, allowed to form callus, and secondary shoots isolated and subcloned. Complete segregation of transformed plastid genome copies (homoplasmicity) in independent subclones is assessed by standard techniques of Southern blotting (Sambrook et al., (1989) Molecular Cloning: A Laboratory Manual, Cold Spring Harbor Laboratory, Cold Spring Harbor). BamHI/EcoRI-digested total cellular DNA (Mettler, I. J. (1987) Plant Molecular Biology Reporter 5, 346349) is separated on 1% Tris-borate (TBE) agarose gels, transferred to nylon membranes (Amersham) and probed with 32P-labeled random primed DNA sequences corresponding to a 0.7 kb BamHI/HindIII DNA fragment from pC8 containing a portion of the plastid targeting sequence. Homoplasmic shoots are rooted aseptically on spectinomycin-containing MS/IBA medium (McBride, K. E. et al. (1994) PNAS USA 91, 7301-7305) and transferred to the greenhouse.

The genetic properties engineered into the transgenic seeds and plants described above are passed on by sexual reproduction or vegetative growth and can thus be maintained and propagated in progeny plants. Generally, maintenance and propagation make use of known agricultural methods developed to fit specific purposes such as tilling, sowing or harvesting.

Use of the advantageous genetic properties of the transgenic plants and seeds according to the invention can further be made in plant breeding. Depending on the desired properties, different breeding measures are taken. The relevant techniques are well known in the art and include but are not limited to hybridization, inbreeding, backcross breeding, multi-line breeding, variety blend, interspecific hybridization, aneuploid techniques, etc. Thus, the transgenic seeds and plants according to the invention can be used for the breeding of improved plant lines that, for example, increase the effectiveness of conventional methods such as herbicide or pesticide treatment or allow one to dispense with said methods due to their modified genetic properties.

While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

The following examples are offered by way of illustration and not by way of limitation.

### EXPERIMENTAL

Standard recombinant DNA and molecular cloning techniques used here are well known in the art and are described by J. Sambrook, E. F. Fritsch and T. Maniatis, Molecular Cloning: A Laboratory manual, Cold Spring Harbor laboratory, Cold Spring Harbor, N.Y. (1989) and by T. J. Silhavy, M. L. Berman, and L. W. Enquist, Experiments with Gene Fusions, Cold Spring Harbor Laboratory, Cold Spring Harbor, N.Y. (1984) and by Ausubel, F. M. et al., Current Protocols in Molecular Biology, published by Greene Publishing Assocation and Wiley-Interscience (1987).

### Example 1

### Biochemically Defined Transit Peptides from Chlamydomonas reinhardtii Targeting the Plastid Stroma

Transit peptides derived from higher plants are not the only molecules capable of directing a foreign protein to the plastid stroma in stably transformed plants. This is based on biological conservation of plastid protein import. The endosymbiotic event that led to plastids occurred long before unicellular and multicellular phototrophs split. During this period endosymbiotic gene transfer from the chloroplast to the nuclear genome led to the acquisition of transit peptides by the transferred genes, and the development of protein import machinery in plastids. It's clear that higher plants possess unique protein import characteristics. However in vitro data show that nuclear encoded plastid proteins from a glaucocystophyte alga like Cyanophora paradoxa (Steiner and Luffelhardt, 2005) or the unicellular alga Chlamydomonas reinhardtii (Mishkind et al., 1985 and Yu et al., 1988) can be imported by plastids derived from higher plants like spinach and pea.

The present invention includes transit peptides from lower photosynthetic organisms. The basis for these transit peptides are that they were identified by N-terminal sequencing of proteins purified from plastids. To define the transit peptide according to the invention and map the cleavage site, the N-terminus of the purified protein was compared to the gene sequence. A transit peptide is the 5′-segment of the open reading frame, from the translation start codon to the N-terminus of the plastid purified protein. Several of these have been identified in Chlamydomonas reinhardtii and are summarized in a review by Franzen et al (1990).

The present invention includes transit peptides derived from the PSI P28, PSI P30, PSI P35, PSI P37, Rubisco SS and ATPase-γ subunit genes from Chlamydomonas reinhardtii that function to target a heterologous protein to plastids in transgenic maize. The present invention further includes transit peptides derived from the Cytochrome c550, Cytochrome c553 and psaF subunit genes from cyanobacteria that function to target a heterologous protein to plastids in transgenic maize. Transit peptides derived from the EPSPS gene from Dunaliella salina function to target a heterologous protein to plastids in transgenic maize. The transit peptides of the invention are depicted in Table 2.

TABLE 2
Transit peptides derived from Chamydomonas reinhardtii,
cyanobacteria and Dualiella salina with
the corresponding SEQ ID NO.

| SEQ ID NO: | Gene name: |
|---|---|
| 2 | PSI P28 |
| 4 | PSI P30 |
| 6 | PSI P35 |
| 8 | PSI P37 |
| 10 | ssRubisco |
| 12 | ATPase gamma |
| 14 | Cytochrome c550 |
| 16 | Cytochrome c553 |
| 18 | psaF |
| 20 | D. salina EPSPS |

Each transit peptide will be back-translated to a maize-optimized using a maize coding sequence table, for example software from Vector NTI. Unless otherwise noted the coding sequence for each transit peptide was assembled by annealing and extending two partially complementary oligonucleotide primers. The chloroplast transit peptides will be linked to the maize optimized AmCyan protein coding sequence, SEQ ID NO: 32. This protein will be used here as a reporter for transit peptide characterization. The transit peptide and trait protein coding sequences will be fused with splicing by overlap extension PCR(SOE-PCR). The PCR product will be digested by the appropriate restriction endonucleases and inserted into an expression cassette. The complete expression cassette, SEQ ID NO: 21, with the addition of the insert will be cloned into an Agrobacterium tumefaciens binary vector containing the FMV 34S-CaMV ³⁵S dual enhancer complex. Each binary vector will be transformed into maize.

### Example 2

### Creation of the Transit Peptides Fused to AmCyan

2A: Creation of P28, P30, P35, P37, ssRubisco, ATPase, c550, c553 and psaF

The following primers were ordered from either Sigma or IDT and used as forward PCR primers (“AmCyan rev” was used as a reverse primer for PCR reactions) to fuse the transit peptides to the maize optimized AmCyan, SEQ ID NO: 32. The primer sequences and corresponding SEQ ID NOs are depicted in Table 3. The AmCyan reverse primer, SEQ ID NO: 31, is used as the reverse primer in the PCR fusing the various CTP to the AmCyan sequence.

TABLE 3
Chloroplast transit peptide primer
sequences

| SEQ ID | Primer | |
|---|---|---|
| NO: | name | Primer sequence 5′ - 3′ |
| 22 | P28 | TATATACCATGGCATTGGTCGCCCGACCAGTTT |
| | | TATCAGCAAGGGTAGCGGCAAGTCGCCCTCGGG |
| | | TAGCTGCCCGTAAAGCTGTGAGAGTTAGCGCGG |
| | | CCCTGAGCAACAAGTTCATCGGCG |
| 23 | P30 | CAAGCTTTGAGTAGCAGGGTGAATATAGCCGCA |
| | | AAACCACAGCGAGCGCAACGTTTAGTAGTTAGA |
| | | GCAGCCCTGAGCAACAAGTTCATCGGCG |
| 24 | P35 | CAAACTTTGGCGAGCCGACCATCATTACGTGCT |
| | | AGTGCCAGGGTCGCGCCTAGACGCGCACCACGG |
| | | GTTGCAGTGGTAACAAAAGCTGCCCTGAGCAAC |
| | | AAGTTCATCGGCG |
| 25 | P37 | CAAGCATTAGCGACTCGACCTTCAGCTATAAGG |
| | | CCAACAAAAGCTGCCCGTAGAAGCAGTGTAGTT |
| | | GTGCGGGCAGCCCTGAGCAACAAGTTCATCGGC |
| | | G |
| 26 | ssRubisco | TATATACCATGGCCGCGGTCATAGCCAAATCAA |
| | | GCGTTTCCGCAGCCGTGGCAAGGCCTGCTAGAT |
| | | CTAGCGTGAGGCCGATGGCTGCCTTGAAACCCG |
| | | CAGTCAAAGCGGCACCTGTGGCAGCTCCAGCAC |
| | | AAGCGAATCAGGCCCTGAGCAACAAGTTCATCG |
| | | GCG |
| 27 | ATPase | TATATACCATGGCCGCTATGTTAGCAAGTAAAC |
| | | AAGGCGCGTTTATGGGAAGGAGTAGCTTCGCAC |
| | | CTGCCCCAAAAGGTGTTGCTAGCAGAGGGTCAT |
| | | TGCAGGTGGTAGCGGCCCTGAGCAACAAGTTCA |
| | | TCGGCG |
| 28 | c550 | AACAAAATACTGGGGATTGATCCACTTAAGAAA |
| | | CCTATCTTTGGAATAAGTGCATTCGTATTACTA |
| | | TTTTGGCAATTGAATGTTGGTGCCGCGAATGCT |
| | | GCCCTGAGCAACAAGTTCATCGGCG |
| 29 | c553 | TTTAAACTGTTTAACCAAGCGAGTAGAATATTC |
| | | TTTGGGATTGCTTTGCCATGTCTTATCTTTTTA |
| | | GGAGGTATATTCAGTTTAGGAAATACAGCCCTA |
| | | GCAGCCCTGAGCAACAAGTTCATCGGCG |
| 30 | psaF | AAACATTTACTTGCGTTGCTGCTAGCCTTTACA |
| | | TTATGGTTCAATTTTGCTCCAAGTGCAAGTGCA |
| | | GCCCTGAGCAACAAGTTCATCGGCG |
| 31 | AmCyan | TATATAGAGCTCTCAGAACG |
| | rev | |

The primers were used in the following PCR mixture:
0.5 μl pDNA template (AmCyan-individual chloroplast transit petide: additional detail are provided below), 1.0 μl forward primer (20 μM), 1.0 μl reverse primer (20 μM), 5.0 μl Extensor green buffer, 2.0 μl Quik Solution™, 1.0 μl dNTPs (10 mM) 0.5 μl PFU Turbo polymerase, 0.5 μl Extensor polymerase blend, 38.5 μl d₂H₂O.
   The following thermocycler program was used:
95° C. for 5 minutes (denaturing), 10 cycles of 95° C. for 1 minutes, 30° C. for 30 seconds, 68° C. for 4 minutes followed by 20 cycles of 95° C. for 30 seconds, 45° C. for 1 minutes, and 68° C. for 4 minutes with a 68° C. final extension for 15 minutes.
   Products were gel purified and cut with Sad or NcoI/SacI and ligated into an appropriately cut backbone vector pSYN15460, SEQ ID NO: 21, as described below. pSYN15460, SEQ ID NO: 21, was cut with NcoI/SacI to prepare for insertion of the following: p28-AmCyan, ssRUBISCO-AmCyan and ATPase-AmCyan. pSYN15460, SEQ ID NO: 21, was cut with NcoI, filled in using T4 DNA polymerase and then cut with Sad to create a blunt/SacI fragment for insertion of the following: c550-AmCyan, c553-AmCyan, psaF-AmCyan, p30-AmCyan, p35-AmCyan, and p37-AmCyan.

2B: Creation of D. salina EPSPS

The following primers were ordered from IDT and used as forward and reverse PCR primers to fuse the transit peptides to the maize optimized AmCyan, SEQ ID NO: 32. Backbone vector pSYN15460, SEQ ID NO: 21, was cut with NcoI, filled in using T4 DNA polymerase and then cut with Sad to create a blunt/SacI fragment for insertion of the D. salina EPSPS-AmCyan reporter gene. An EPSPS overlap sequence was added to AmCyan via PCR using the primers, SEQ ID NO: 31 and 34. The primer sequences and corresponding SEQ ID NOs are depicted in Table 4.

TABLE 4
Chloroplast transit peptide primer sequences

| SEQ ID | Primer | |
|---|---|---|
| NO: | name | Primer sequence 5′ - 3′ |
| 31 | AmCyan | TATATAGAGCTCTCAGAACG |
| | rev | |
| 33 | EPSPS | CTGGCCAGGCAGGGCGGCAGCCTGAGGGCCAGCCA |
| | CTP | GTGCAACGCCGGCCTGGCCAGGAGGGTGGAGGTGG |
| | forward | GCGCCCTGGTGGTGCCGAGGCCGATCAGCGTGAAC |
| | | GACGTGGTGCCGCACGTGTACAGCGCCCCGCTGAG |
| | | CG |
| 34 | EPSPS | TGCTCAGGGCCGGGGCGCTGTGGGCGGCCAGGGTG |
| | CTP | GCGCTGCACACGGTGGTCTGCAGCCTCCTGGTGCT |
| | reverse | CCTGATGCTGCTCTTGCTGCAGCTCCTCCTGGCCA |
| | | CGCTCAGCGGGGCGCTGTACACGTGCGGCACCACG |
| | | TCGTT |
| 35 | AmCyan | CAGCGCCCCGGCCCTGAGCA |
| | w/EPSPS | |
| | forward | |

The primers were used in the following mixture:
1.0 μl pDNA template (AmCyan-EPSPS), 1.0 μl forward primer (20 μM), 1.0 μl reverse primer (20 μM), 2.5 μl Pfu buffer, 1.0 μl dNTPs (25 mM), 1.0 μl PFU Turbo polymerase, 17.5 μl d₂H₂O.
   The following thermocycler program was used:
95° C. for 2 minutes (denaturing), 30 cycles of 95° C. for 1 minutes, 45° C. for 1 minute and 68° C. for 4 minutes with a 68° C. final extension for 15 minutes.
   The PCR product was gel purified and combined with the D. salina EPSPS transit peptide sequence in equimolar concentration. The following primers, SEQ ID NO: 31 and 34, were used the PCR mixture according to the following conditions.
   The primers were used in the following mixture:
1.0 μl pDNA template (AmCyan w/EPSPS overlap), 1.0 μl dsDNA template (EPSPS transit peptide) 1.0 μl forward primer (20 μM), 1.0 μl reverse primer (20 μM), 2.5 μl Pfu buffer, 1.0 μl dNTPs (25 mM), 1.0 μl PFU Turbo polymerase, 16.5 μl d₂H₂O.
   The following thermocycler program was used:
95° C. for 2 minutes (denaturing), 30 cycles of 95° C. for 1 minutes, 45° C. for 1 minute and 68° C. for 4 minutes with a 68° C. final extension for 15 minutes.
   The PCR product was gel purified, cut with SacI, ligated to the pSYN15460 backbone, SEQ ID NO: 21.

Completed vectors comprising the insertion, Examples 2A and 2B, were transformed into DH5α competent cells. Then, the transformed DH5α cells are used to make a glycerol stock suitable for long term storage. These cells are used to inoculate and grow liquid cultures suitable for DNA extraction. The extracted DNA derived from this glycerol stock is the basis for subsequent cloning into the Agrobacterium tumefaciens binary vector.

### Example 3

### Chloroplast Isolation

Maize chloroplasts were isolated from maize plants that were at the V2-V3 stage. Isolation was adapted from Asakura et al. (Plant Cell 16(1): 201-214 2004). Plants were grown in soil in a greenhouse until isolation, they were then placed in a dark chamber for a period of 3-5 hours to decrease photosynthesis to eliminate starch in the preparations. Leaves were removed and midveins were removed from the leaves. All subsequent steps were done on ice. The leaves were cut into smaller pieces, typically in a total of 15 g amounts. The cut plant pieces were placed in an ice cold grind buffer containing 50 mM HEPES, pH 7.6, 0.33M sorbitol, 1 mM MgCl₂, 2 mM ETDA, and 1% (w:v) BSA at a 1:4 (w:v) ratio. Plants were homogenized to a fine mince in cold buffer lightly to release the chloroplasts either via a blender or via hand with a sharp razor blade on ice in a polypropylene tube to try and reduce crushing of the chloroplasts. After homogenization, the homogenate was filtered through two layers of Miracloth into a beaker on ice. This filtrate was then filtered once more through 4 layers of Miracloth. This filtrate was placed in a 50 mL polycarbonate tube and centrifuged at 4,000×g, 4° C., for 8 minutes and the supernatant was discarded. The pellet was resuspended in 1-2 mL resuspension (RB) buffer (50 mM HEPES, pH 7.6, 0.33M sorbitol, 1 mM DTT) and layered onto a 12.5 mL 30% (v:v) percoll gradient containing 50 mM HEPES, pH 7.6, 0.33M sorbitol, and 5 mM DTT. The gradient was then centrifuged in a 50 mL polycarbonate tube in a clinical centrifuge, swinging bucket attachment, at 1350×g, 4° C. for 12 minutes. The pellet contained the chloroplasts and the supernatant, which contained broken chloroplast material, was discarded; the pellet was resuspended in 0.5 mL RB. Protein quantitation was performed of the resuspended pellet via the Bradford (Analytical Biochemistry 72: 248-254 1976) method. A small aliquot of the resuspended pellet was also visualized on a Leica DMLB light microscope at 40 and 100× to examine chloroplasts for intactness.

Tobacco isolations were based upon protocols by Spencer and Wildman (Biochemistry. 3(7): 954-959 1964). Tobacco was isolated from young plants grown in soil that had 4-5 small leaves. Typically plants were isolated in 4-5 g batches. Leaves were cut into smaller pieces and then placed in an ice cold grind buffer containing 50 mM HEPES, pH 7.6, 0.33M sorbitol, 1 mM MgCl₂, 2 mM ETDA, and 1% (w:v) BSA at a 1:4 (w:v) ratio. Plants were homogenized to a fine mince either via a blender or via hand with a sharp razor blade on ice in a polypropylene tube to try and reduce crushing of the chloroplasts. The homogenate was then filtered through 2 layers of Miracloth into a beaker on ice. This filtrate was again filtered once more through 4 layers of Miracloth. The filtrate was placed in a 50 mL polycarbonate tube and centrifuged at 1000×g, 4° C., for 15 minutes. The supernatant was discarded and the pellet, which contained chloroplasts was resuspended in 0.5 mL RB. Protein quantitation was performed of the resuspended pellet via the Bradford (Analytical Biochemistry 72: 248-254 1976) method. A small aliquot of the resuspended pellet was also visualized on a Leica DMLB light microscope at 40 and 100× to visualize chloroplasts for intactness.

### Example 4

### Tobacco Transcient Assays

To determine if heterologous proteins encoded by peptide constructs could be incorporated into chloroplasts in a transient system, the peptide constructs were transformed into Agrobacterium (LBA4404 strain). The Agrobacterium harboring the peptide construct of interest was grown in YP media overnight, 28° C., and then centrifuged 5000 rpm for ten minutes. The pellet was resusupended in MS inducing media (0.44% (w:v) MS basal salt mixture, 2% sucrose (w:v) and allowed to induce for 2 hours at room temperature. Young tobacco plants (having only 3-4 leaves) that were grown in soil were then infiltrated with the induced Agrobacterium by injecting the underside of leaves with a blunt ended syringe. Infiltration could be seen by eye as the liquid traveled into the leaf. Plants that were infiltrated were allowed to grow under normal greenhouse conditions for 3-4 days after infiltration before tissue was harvested for chloroplast isolations.

Reactions from in vitro recombinant peptide-AmCyan importation studies with maize chloroplasts were treated with thermolysin as stated above and then resuspended in RB. Small aliquots of the reactions (10 μL) were put under a Zeiss confocal microscope with filters applied at 486 and 594 to visualize AmCyan and autofluorescence of chloroplasts respectively. Overlays were done to determine if the protein was imported into the chloroplast.

The peptides, P28, P35, P37, ATPase and SSRubisco, were derived from Chlamydomonas reinhardtii sequences and were shown according to the invention to enable heterologous protein import into chloroplasts. Of these peptides fused to AmCyan, all five of them allow transport of AmCyan into the chloroplast in in vitro chloroplast importation assays. The remaining examples are in progress experimentally. Thus, the following peptides, P30, cytochrome c₅₅₀, cytochrome c₅₅₃, psaF and D. salina EPSPS receive a place holder of N/A, meaning not applicable. These results are depicted in Table 5.

TABLE 5
Transient assay results

| Transit Peptide | AmCyan |
|---|---|
| P28 | Positive |
| P30 | N/A |
| P35 | Positive |
| P37 | Positive |
| ssRubisco | Positive |
| ATPase | Positive |
| cytochrome c₅₅₀ | N/A |
| cytochrome c₅₅₃ | N/A |
| psaF | N/A |
| D. salina EPSPS | N/A |

TABLE 6
Includes data for P28, P35, P37, SSR, ATPase transit peptides,
confirming positive chloroplast targeting as shown in Table 5,
and further includes data for remaining transit peptides C553,
C550, P30, PSAF, EPSPS, PSAF.

| | Maize | | | Tobacco | | |
|---|---|---|---|---|---|---|
| Transit Peptide | Low | Medium | High | Low | Medium | High |
| P28 | | | x | | | x |
| C553 | X | | | | x | |
| C550 | X | | | x | | |
| ATPase | | x | | | | x |
| P30 | | x | | | x | |
| P35 | | | x | | x | |
| SSR | | | x | | | x |
| P37 | | | x | | x | |
| PSAF | X | | | | x | |
| EPSPS | X | | | x | | |

All tobacco constructs were analyzed at T0 stage. All maize constructs were analyzed at T1, with the exception of SSR and EPSPS, which were analyzed at T0 stage. All targeting capacity estimations were based on confocal microscopy of chloroplast fractions, where visualization of the AmCyan reporter protein expression was performed.

### Example 5

### Methods for Determining Chloroplast Targeting in Transgenic Plants

5.A. Maize Transformation:

Transformation of immature maize embryos will be performed essentially as described in Negrotto et al., Plant Cell Reports 19:798-803 (2000). Various media constituents described therein can be substituted.

Agrobacterium strain LBA4404 (Invitrogen) containing the plant transformation plasmid is grown on YEP (yeast extract (5 g/L), peptone (10 g/L), NaCl (5 g/L),15 g/L agar, pH 6.8) solid medium for 2 to 4 days at 28° C. Approximately 0.8×10⁹ Agrobacteria are suspended in LS-inf media supplemented with 100 μM acetosyringone (As) (LSAs medium) (Negrotto et al., Plant Cell Reports 19:798-803 (2000)). Bacteria are pre-induced in this medium for 30-60 minutes.

Immature embryos from maize line, A188, or other suitable maize genotypes are excised from 8-12 day old ears into liquid LS-inf+100 μM As (LSAs). Embryos are vortexed for five (5) seconds and rinsed once with fresh infection medium. Infection media is removed and Agrobacterium solution is then added and embryos are vortexed for 30 seconds and allowed to settle with the bacteria for 5 minutes. The embryos are then transferred scutellum side up to LSAs medium and cultured in the dark for two to three days. Subsequently, between 20 and 25 embryos per petri plate are transferred to LSDc medium supplemented with cefotaxime (250 mg/L) and silver nitrate (1.6 mg/L) (Negrotto et al., Plant Cell Reports 19:798-803 (2000)) and cultured in the dark for 28° C. for 10 days.

Immature embryos producing embryogenic callus are transferred to LSD1M0.5S medium (LSDc with 0.5 mg/L2,4-D instead of Dicamba, 10 g/L mannose, 5 g/L sucrose and no silver nitrate). The cultures are selected on this medium for 6 weeks with a subculture step at 3 weeks. Surviving calli are transferred either to LSD1M0.5S medium to be bulked-up or to Reg1 medium (as described in Negrotto et al., Plant Cell Reports 19:798-803 (2000). Following culturing in the light (16 hour light/8 hour dark regiment), green tissues are then transferred to Reg2 medium without growth regulators (as described in Negrotto et al., Plant Cell Reports 19:798-803 (2000) and incubated for 1-2 weeks. Plantlets are transferred to Magenta GA-7 boxes (Magenta Corp, Chicago Ill.) containing Reg3 medium, as described in Negrotto et al. 2000, and grown in the light. Plants that are PCR positive for any component of the expression cassette are transferred to soil and grown in the greenhouse. The presence of any component of the expression cassette is determined by +/−PCR assay or by a Taqman copy number assay.

One skilled in the art will recognize maize transformation as an example of a monocot crop species. The results of this experimentation serve as an example extendible to other monocot species, such as rice, wheat, barley and sugarcane.

5.B. Tobacco Transformation:

Agrobacterium tumefaciens strain LBA4404 containing a transformation vector containing an expression cassette will be used to infect leaf explants of Nicotiana tabacum c.v. Petit Havana (SR1). Initially, the tobacco leaves are cut into 1-2 mm wide slices, exposed to the Agrobacterium for 5 minutes, and are placed on sterile paper to blot away excess liquid and then placed on co-cultivation medium for 3 days. The leaf slices are moved to selection/regeneration medium containing the appropriate selection agent. Selection agent resistant shoots are transplanted to soil. The plants are selfed or outcrossed with pollens from nontransgenic SR1 plants to produce seeds. The presence of any component of the expression cassette is determined by +/−PCR assay or by a Taqman copy number assay.

One skilled in the art will recognize tobacco transformation as an example of a dicot crop species. The results of this experimentation serve as an example extendible to other dicot species, such as soybean, sugarbeet, tomato and sunflower.

All publications and patent applications mentioned in the specification are indicative of the level of those skilled in the art to which this invention pertains. Although the foregoing invention has been described in some detail by way of illustration and example for purposes of clarity of understanding, it will be obvious that certain changes and modifications may be practiced within the scope of the appended claims.

What is claimed:

1. A plant having stably incorporated into its genome a DNA construct, said construct comprising a nucleotide sequence encoding a plastid targeting peptide wherein said nucleotide sequence is operably linked to a nucleotide sequence encoding a polypeptide of interest, wherein said nucleotide sequence encoding a plastid targeting peptide is selected from the group consisting of:
a) a nucleotide sequence set forth in SEQ ID NOS: 1, 3, 5, 7, 9, 11, 13, 15, 17 and 19;
b) a nucleotide sequence having at least 90% sequence identity to SEQ ID NOS: 1, 3, 5, 7, 9, 11, 13, 15, 17 and 19, wherein said nucleotide sequence encodes a peptide capable of transporting an operably linked polypeptide to a plastid;
c) a nucleotide sequence encoding the amino acid sequence set forth in SEQ ID NOS: 2, 4, 6, 8, 10, 12, 14, 16, 18 and 20; and,
d) a nucleotide sequence encoding an amino acid sequence having at least 90% sequence identity to SEQ ID NOS: 2, 4, 6, 8, 10, 12, 14, 16, 18 and 20 wherein said amino acid sequence is capable of transporting an operably linked polypeptide to a plastid.

2. The plant of claim 1, wherein said plant is a monocotyledonous plant.

3. The plant of claim 2, wherein said monocotyledonous plant is a maize, rice or sugarcane plant.

4. The plant of claim 1, wherein said plant is a dicotyledonous plant.

5. The plant of claim 4, wherein said dicotyledonous plant is a soybean plant.

6. A method of expressing a polypeptide of interest in a plant, said method comprising introducing into said plant a DNA construct comprising a nucleotide sequence encoding a plastid targeting peptide, wherein said nucleotide sequence encoding a plastid targeting peptide is operably linked to a nucleotide sequence encoding said polypeptide of interest, said nucleotide sequence encoding a plastid targeting peptide is selected from the group consisting of:
a) a nucleotide sequence set forth in SEQ ID NOS: 1, 3, 5, 7, 9, 11, 13, 15, 17 and 19;
b) a nucleotide sequence having at least 90% sequence identity to SEQ ID NOS: 1, 3, 5, 7, 9, 11, 13, 15, 17 and 19, wherein said nucleotide sequence encodes a peptide capable of transporting an operably linked polypeptide to a plastid;
c) a nucleotide sequence encoding the amino acid sequence set forth in SEQ ID NOS: 2, 4, 6, 8, 10, 12, 14, 16, 18 and 20; and,
d) a nucleotide sequence encoding an amino acid sequence having at least 90% sequence identity to SEQ ID NOS: 2, 4, 6, 8, 10, 12, 14, 16, 18 and 20 wherein said amino acid sequence is capable of transporting an operably linked polypeptide to a plastid.

7. The method of 6, wherein said plant is a monocotyledonous plant.

8. The method of 6, wherein said plant is a dicotyledonous plant.

9. The method of 6, wherein said polypeptide sequence of interest is a bioprocessing enzyme.

10. The method of 6, wherein said nucleotide sequence encoding said polypeptide of interest is a herbicide tolerant gene.

11. A substantially purified peptide capable of transporting a polypeptide into a cellular plastid wherein said peptide comprises an amino acid sequence set forth in the group consisting of SEQ ID NOS: 1, 3, 5, 7, 9, 11, 13, 15, 17 and 19 and variants thereof, wherein said variant retains the ability to transport a polypeptide.

12. The peptide of claim 11, wherein said peptide is operably linked to a polypeptide of interest.

13. A nucleotide sequence that encodes one of the peptides of any one of claims 11-12.

14. A nucleotide sequence having 90% sequence identity to a nucleotide sequence of claim 13.

15. A DNA construct comprising the nucleotide sequence of claim 13 or 14.

16. A plant cell having stably incorporated into its genome a DNA construct, said construct comprising a nucleotide sequence encoding a plastid targeting peptide wherein said nucleotide sequence is operably linked to a nucleotide sequence encoding a polypeptide of interest, wherein said nucleotide sequence encoding a plastid targeting peptide is selected from the group consisting of:
a) a nucleotide sequence set forth in SEQ ID NOS: 2, 4, 6, 8, 10, 12, 14, 16, 18 and 20;
b) a nucleotide sequence having at least 90% sequence identity to SEQ ID NOS: 1, 3, 5, 7, 9, 11, 13, 15, 17 and 19, wherein said nucleotide sequence encodes a peptide capable of transporting an operably linked polypeptide to a plastid;
c) a nucleotide sequence encoding the amino acid sequence set forth in SEQ ID NOS: 2, 4, 6, 8, 10, 12, 14, 16, 18 and 20; and,
d) a nucleotide sequence encoding an amino acid sequence having at least 90% sequence identity to SEQ ID NOS: 2, 4, 6, 8, 10, 12, 14, 16, 18 and 20 wherein said amino acid sequence is capable of transporting an operably linked polypeptide to a plastid.

17. The plant cell of claim 16, wherein said plant cell is from a dicotyledonous plant.

18. The plant cell of claim 16, wherein said plant cell is from a monocotyledonous plant.

19. A plant comprising the plant cell of claim 18.

20. Stably transformed seed of the plant of claim 19.


SEQUENCE LISTING (32 sequences)

SEQ ID NO: 1 - DNA, 90 bp, Chlamydomonas reinhardtii
atggcattggtcgcccgaccagttttatcagcaagggtagcggcaagtcgccctcgggtagctgcccgtaaagctgtgagagttagcgcg

SEQ ID NO: 2 - protein, 30 aa, Chlamydomonas reinhardtii
MALVARPVLSARVAASRPRVAARKAVRVSA

SEQ ID NO: 3 - DNA, 72 bp, Chlamydomonas reinhardtii
atgcaagctttgagtagcagggtgaatatagccgcaaaaccacagcgagcgcaacgtttagtagttagagca

SEQ ID NO: 4 - protein, 24 aa, Chlamydomonas reinhardtii
MQALSSRVNIAAKPQRAQRLVVRA

SEQ ID NO: 5 - DNA, 90 bp, Chlamydomonas reinhardtii
atgcaaactttggcgagccgaccatcattacgtgctagtgccagggtcgcgcctagacgcgcaccacgggttgcagtggtaacaaaagct

SEQ ID NO: 6 - protein, 30 aa, Chlamydomonas reinhardtii
MQTLASRPSLRASARVAPRRAPRVAVVTKA

SEQ ID NO: 7 - DNA, 78 bp, Chlamydomonas reinhardtii
atgcaagcattagcgactcgaccttcagctataaggccaacaaaagctgcccgtagaagcagtgtagttgtgcgggca

SEQ ID NO: 8 - protein, 26 aa, Chlamydomonas reinhardtii
MQALATRPSAIRPTKAARRSSVVVRA

SEQ ID NO: 9 - DNA, 135 bp, Chlamydomonas reinhardtii
atggccgcggtcatagccaaatcaagcgtttccgcagccgtggcaaggcctgctagatctagcgtgaggccgatggctgccttgaaacccgcagtcaaagcggcacctgtggcagctccagcacaagcgaatcag

SEQ ID NO: 10 - protein, 45 aa, Chlamydomonas reinhardtii
MAAVIAKSSVSAAVARPARSSVRPMAALKPAVKAAPVAAPAQANQ

SEQ ID NO: 11 - DNA, 105 bp, Chlamydomonas reinhardtii
atggccgctatgttagcaagtaaacaaggcgcgtttatgggaaggagtagcttcgcacctgccccaaaaggtgttgctagcagagggtcattgcaggtggtagcg

SEQ ID NO: 12 - protein, 35 aa, Chlamydomonas reinhardtii
MAAMLASKQGAFMGRSSFAPAPKGVASRGSLQVVA

SEQ ID NO: 13 - DNA, 102 bp, Synechococcus sp. PCC 7002
atgaacaaaatactggggattgatccacttaagaaacctatctttggaataagtgcattcgtattactattttggcaattgaatgttggtgccgcgaatgct

SEQ ID NO: 14 - protein, 34 aa, Synechococcus sp. PCC 7002
MNKILGIDPLKKPIFGISAFVLLFWQLNVGAANA

SEQ ID NO: 15 - DNA, 105 bp, Synechocystis sp. PCC 6803
atgtttaaactgtttaaccaagcgagtagaatattctttgggattgctttgccatgtcttatctttttaggaggtatattcagtttaggaaatacagccctagca

SEQ ID NO: 16 - protein, 35 aa, Synechocystis sp. PCC 6803
MFKLFNQASRIFFGIALPCLIFLGGIFSLGNTALA

SEQ ID NO: 17 - DNA, 69 bp, Synechocystis sp. PCC 6803
atgaaacatttacttgcgttgctgctagcctttacattatggttcaattttgctccaagtgcaagtgca

SEQ ID NO: 18 - protein, 23 aa, Synechococcus sp. PCC 7002
MKHLLALLLAFTLWFNFAPSASA

SEQ ID NO: 19 - DNA, 237 bp, Dunaliella salina
atgctggcgaggcagggtggctctctcagggcctcgcagtgtaatgcagggctggctaggagggtcgaggtgggcgccctggttgtgccacgtcccatctcggtaaatgacgtggtccctcatgtgtacagcgcccccctttcggtggcgaggagaagttgcagcaagagcagcatcaggagcacgaggcgcctgcagacgacagtttgctctgctactttggcggctcacagcgcc

SEQ ID NO: 20 - protein, 79 aa, Dunaliella salina
MLARQGGSLRASQCNAGLARRVEVGALVVPRPISVNDVVPHVYSAPLSVARRSCSKSSIRSTRRLQTTVCSATLAAHSA

SEQ ID NO: 21 - DNA, 4145 bp, Artificial
  Cloning Vector Sequence
ccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacggggacccaagctttggcagacaaagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtggatccccaccatggcgcccaccgtgatgatggcctcgtcggccaccgccgtcgctccgttccaggggctcaagtccaccgccagcctccccgtcgcccgccgctcctccagaagcctcggcaacgtcagcaacggcggaaggatccgctgcatgagctcttcatatgacgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgcggaccgcagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttcgaccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcacgatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcctttttttctgatcaaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 22 - DNA, 123 bp, Artificial
  Photosystem I P28 including primer
tatataccatggcattggtcgcccgaccagttttatcagcaagggtagcggcaagtcgccctcgggtagctgcccgtaaagctgtgagagttagcgcggccctgagcaacaagttcatcggcg

SEQ ID NO: 23 - DNA, 94 bp, Artificial
  Photosystem I P30 including primer
caagctttgagtagcagggtgaatatagccgcaaaaccacagcgagcgcaacgtttagtagttagagcagccctgagcaacaagttcatcggcg

SEQ ID NO: 24 - DNA, 112 bp, Artificial
  Photosystem I P35 including primer
caaactttggcgagccgaccatcattacgtgctagtgccagggtcgcgcctagacgcgcaccacgggttgcagtggtaacaaaagctgccctgagcaacaagttcatcggcg

SEQ ID NO: 25 - DNA, 100 bp, Artificial
  Photosystem I P37 including primer
caagcattagcgactcgaccttcagctataaggccaacaaaagctgcccgtagaagcagtgtagttgtgcgggcagccctgagcaacaagttcatcggcg

SEQ ID NO: 26 - DNA, 168 bp, Artificial
  SSRubisco including primer
tatataccatggccgcggtcatagccaaatcaagcgtttccgcagccgtggcaaggcctgctagatctagcgtgaggccgatggctgccttgaaacccgcagtcaaagcggcacctgtggcagctccagcacaagcgaatcaggccctgagcaacaagttcatcggcg

SEQ ID NO: 27 - DNA, 138 bp, Artificial
  ATPase including primer
tatataccatggccgctatgttagcaagtaaacaaggcgcgtttatgggaaggagtagcttcgcacctgccccaaaaggtgttgctagcagagggtcattgcaggtggtagcggccctgagcaacaagttcatcggcg

SEQ ID NO: 28 - DNA, 124 bp, Artificial
  Cytochrome c550 including primer
aacaaaatactggggattgatccacttaagaaacctatctttggaataagtgcattcgtattactattttggcaattgaatgttggtgccgcgaatgctgccctgagcaacaagttcatcggcg

SEQ ID NO: 29 - DNA, 127 bp, Artificial
  Cytochrome c553 including primer
tttaaactgtttaaccaagcgagtagaatattctttgggattgctttgccatgtcttatctttttaggaggtatattcagtttaggaaatacagccctagcagccctgagcaacaagttcatcggcg

SEQ ID NO: 30 - DNA, 91 bp, Artificial
  psaF including primer
aaacatttacttgcgttgctgctagcctttacattatggttcaattttgctccaagtgcaagtgcagccctgagcaacaagttcatcggcg

SEQ ID NO: 31 - DNA, 20 bp, Artificial
  AmCyan reverse primer
tatatagagctctcagaacg

SEQ ID NO: 32 - DNA, 154 bp, Artificial
  Maize optimized AmCyan
gccctgagcaacaagttcatcggcgacgacatgaagatgacctaccacatggacggctgcgtgaacggccactacttcaccgtgaagggcgagggcagcggcaagccatacgagggcacccagaccagcaccttcaaggtgacgatggccaacg